(19) United States
(12) Patent Application Publication
(10) Pub. No.: US 2026/0273209 A1
(43) Pub. Date: Sep. 17, 2026

(54) PATIENT SENSING FOR RESPIRATORY THERAPY

(71) Applicants: RESMED PTY LTD, Bella Vista, New South Wales (AU); RESMED SENSOR TECHNOLOGIES LIMITED, Dublin (IE)

(72) Inventors: James SUNG, Bella Vista, New South Wales (AU); Andrew William GILLETT, Bella Vista, New South Wales (AU); Liam HOLLEY, Bella Vista, New South Wales (AU); Redmond SHOULDICE, Dublin (IE)

(21) Appl. No.: 19/505,858

(22) PCT Filed: Jun. 27, 2024
(86) PCT No.: PCT/AU2024/050687

§ 371 Date: Jan. 27, 2026

(30) Foreign Application Priority Data
Jun. 28, 2023 (AU) 2023902047

(51) Int. Cl. A61M 16/00; A61M 16/06

(52) U.S. Cl. CPC A61M 16/022; A61M 16/0051; A61M 16/0057; A61M 16/0683; A61M 2205/18; A61M 2205/3303; A61M 2205/50; A61M 2205/502

(57) ABSTRACT

A wearable device for use with a respiratory therapy system is disclosed. The device comprises a body configured to locate with respect to an ear of a patient under respiratory therapy. The patient tracker includes at least one sensor to obtain sensor data of the patient when so located. The wearable device can be configured to provide sensor data to a processor to aid in respiratory therapy of the patient under the respiratory therapy system. A treatment system and method of determining an adjustment to a respiratory therapy for a patient is also disclosed.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

### 1.2.3.2 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal-forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 1.2.3.3 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534.

Another technique is the use of one or more straps and/or stabilising harnesses. Many such harnesses suffer from being one or more of ill-fitting, bulky, uncomfortable and awkward to use.

### 1.2.3.4 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

1.2.3.4.1 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 1.2.3.5 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices (one specimen only, measured using test method specified in ISO 3744 in CPAP mode at 10 cmH2O).

| | A-weighted sound | Year |
|---|---|---|
| RPT Device name | pressure level dB(A) | (approx.) |
| C-Series Tango ™ | 31.9 | 2007 |
| C-Series Tango ™ with Humidifier | 33.1 | 2007 |
| S8 Escape ™ II | 30.5 | 2005 |
| S8 Escape ™ II with H4i ™ Humidifier | 31.1 | 2005 |
| S9 AutoSet ™ | 26.5 | 2010 |
| S9 AutoSet ™ with H5i Humidifier | 28.6 | 2010 |

One known RPT device used for treating sleep disordered breathing is the S9 Sleep Therapy System, manufactured by ResMed Inc. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

An aspect of certain forms of the present technology is a medical device that is easy to use, e.g. by a person who does not have medical training, by a person who has limited dexterity, vision or by a person with limited experience in using this type of medical device.

One form of the present technology comprises a wearable device for use with a respiratory therapy system. The wearable device comprises a body configured to locate with respect to an ear of a patient under respiratory therapy. The device includes at least one sensor to obtain physiological data of the patient when so located. The device can be configured to provide sensor data to a processor to aid in respiratory therapy of the patient under the respiratory therapy system.

An aspect of the present technology is that the at least one sensor may be configured to obtain physiological data of the patient independently of the respiratory therapy system. That is, the at least one sensor of the device does not rely on the respiratory therapy system to acquire the physiological data.

An aspect of the present technology is that the wearable device is able to be worn by the patient independently of the respiratory therapy system and the at least one sensor is configured to acquire the physiological data during an off therapy period.

An off therapy period may be when the patient is not undergoing respiratory therapy or adhering to respiratory therapy. This may include when a patient is in an awake state when the respiratory therapy is applied whilst the patient is sleeping and a period prior to or following a therapy session with a respiratory therapy device. This may enable the obtainment of physiological data that may indicate changes pre and post therapy indicating the efficacy of the therapy.

An aspect of the present technology is that the at least one sensor is further configured to acquire physiological data during an on therapy period.

An aspect of the present technology is that the sensor data may further comprise activity data of the patient.

An aspect of the present technology is that the sensor data further comprises environmental data surrounding the patient.

An aspect of the present technology is that the at least one sensor may be configured to provide sensor data to a processor of the respiratory therapy system. The processor of the respiratory therapy system may be contained in a flow generator having a control system. That is, said processor may be part of the control system of the flow generator.

An aspect of the present technology is that the sensor data may be provided to the processor to effect change to the respiratory therapy. For example, a flow of air delivered to the patient by the respiratory therapy system may be changed, e.g., increased.

An aspect of the present technology is that the sensor data may be provided to the processor to determine one or more characteristics of the patient related to the respiratory therapy. The sensor data of the patient may include activities undertaken by the patient, e.g., a distance run, and also physiological conditions of the patient, e.g., heart rate.

An aspect of the present technology is that the processor may be configured to determine a correlation between the physiological data of the patient and the respiratory therapy of the patient. In some forms, the physiological data of the patient may include the physiological characteristics of the patient set forth above. The correlation may be communicated to the patient to aid in the respiratory therapy of the patient.

An aspect of the present technology is that the sensor data may be provided to the processor to determine one or more characteristics of an environment surrounding the patient related to the respiratory therapy.

An aspect of the present technology is that the body of the wearable device may be configured to locate with respect to an external auditory canal of the patient.

An aspect of the present technology is that the at least one sensor may be configured with respect to the body to be: a) in contact with the patient's skin for collecting the physiological data of the patient; and/or b) spaced from the patient's skin for collecting environmental data related to the patient.

An aspect of the present technology is that the wearable device may further comprise an audio unit operable to selectively generate at least one sound.

An aspect of the present technology is that the device may further comprise an ear hook configured to locate with respect to the patient's external ear, e.g., behind the patients' ear.

An aspect of the present technology is that the ear hook may include the at least one sensor.

One form of the present technology comprises a treatment system comprising a respiratory system. The respiratory system comprises a flow generator configured to generate a flow of air. The respiratory system can comprise a patient interface constructed and arranged to form a seal with a region of the patient's face surrounding an entrance to the patient's airways. The patient interface can be configured to deliver the pressurised or breathable air to the patient's airways for respiratory therapy. The respiratory system can comprise and an air delivery tube coupled between the flow generator and the patient interface to deliver the flow of air from the flow generator to the patient interface as the pressurised or breathable air. The treatment system can also comprise a wearable device configured to locate with respect to an ear of the patient. The wearable device can be as set forth above. The device can be configured to provide sensor data to a processor to aid in respiratory therapy of the patient under the respiratory system.

An aspect of the present technology is that the respiratory system may further comprise a processor to interact with the patient tracker.

An aspect of the present technology is that the respiratory system processor may be configured to control an operation of the respiratory system responsive to the sensor data of the wearable device.

An aspect of the present technology is that the sensor data may be provided to the respiratory system processor to determine one or more characteristics of the patient. That is, the respiratory system processor may be used instead of a processor in the wearable device to determine the one or more characteristics of the patient.

An aspect of the present technology is that the respiratory system processor may be arranged in the flow generator.

An aspect of the present technology is that the respiratory system processor may control an operation of the flow generator.

An aspect of the present technology is that the flow generator may comprise a display interface. The respiratory system processor may be configured to generate an alert to be displayed on the display interface based on sensor data of the at least one sensor of the wearable device.

An aspect of the present technology is that the wearable device may be configured to provide sensor data to a processor of at least one supplementary computing device. The at least one supplementary computing device may comprise a display interface. The at least one supplementary computing device may be configured to generate an alert to be displayed on the display interface of the supplementary computing device based on the sensor data of the wearable device.

An aspect of the present technology is that the alert may be a correlation between the one or more characteristics of the patient and the respiratory therapy of the patient. The correlation may be configured to aid in the respiratory therapy of the patient.

One form of the present technology comprises respiratory therapy system for delivery of respiratory therapy to a patient. The system may comprise one or more processors; and a memory having stored therein a therapy control program for execution by the processor, the therapy control program being configured to control operation of the respiratory therapy system. The therapy control program, when executed by the one or more processors, may cause the one or more processor to receive data from a wearable device when in data communication with the wearable device, the wearable device being configured to acquire sensor data including physiological data of the patient when so located; the wearable device being able to be worn by the patient independently of the respiratory therapy system and being configured to acquire the physiological data during an off therapy period. The therapy control system may determine an adjustment to the respiratory therapy on the basis of the received data, store the determined adjustment in the memory; and apply the adjustment to the respiratory therapy during a therapy session.

An aspect of the present technology is that the wearable device may be configured to be located with respect to an ear of the patient.

An aspect of the present technology is that the therapy control program, when executed by the one or more processors, can cause the one or more processor to determine a correlation between the received data and a data in relation to the respiratory therapy.

An aspect of the present technology is that the received data may comprise an analysis result from analysing the sensor data.

An aspect of the present technology is that the determination of the adjustment is at least partially based on a comparison between received data corresponding to sensor data acquired over a first time period with received data corresponding to sensor data acquired over a second time period. The first time period may be during the off-therapy period, and a second time period may be during an on-therapy period.

An aspect of the present technology is that sensor data may further comprise activity data of the patient and/or environmental data.

An aspect of the present technology is that the therapy control program, when executed by the one or more processors, causes the one or more processor to store the received data in the memory for a predetermined period of time, prior to determining the adjustment.

One form of the present technology comprises a method of determining an adjustment to a respiratory therapy for a patient, comprising acquiring sensor data during an off therapy period using one or more sensors in a wearable device adapted to be located in an ear of the patient, storing the acquired sensor data in a memory device, and determining an analysis data comprising one or more physiological or activity parameters, based on the stored data. The method may include determining the adjustment on the basis of the analysis data.

An aspect of the present technology is that the method may acquire a first sensor data during a first period of time and acquire a second sensor data during a second period of time. The analysis data may be determined by comparing the first sensor data with the second data, or comparing values of one or more parameters calculated from the first sensor data against values of the parameters calculated from the second sensor data. The first time period may be during the off-therapy period, and a second time period may be during an on-therapy period.

An aspect of the present technology is that determining the analysis data may comprise modulating the physiological or activity parameters based on one or more environmental data.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

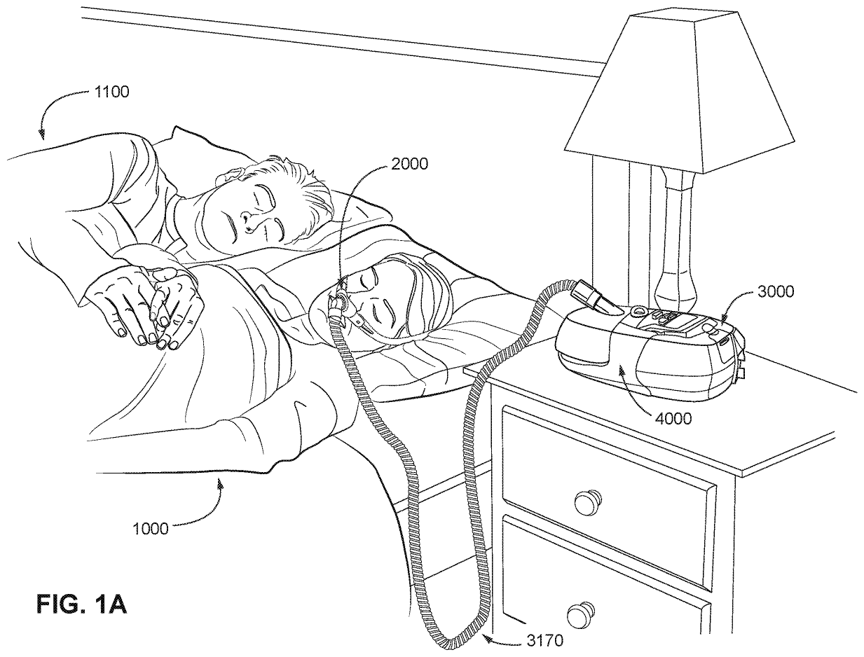

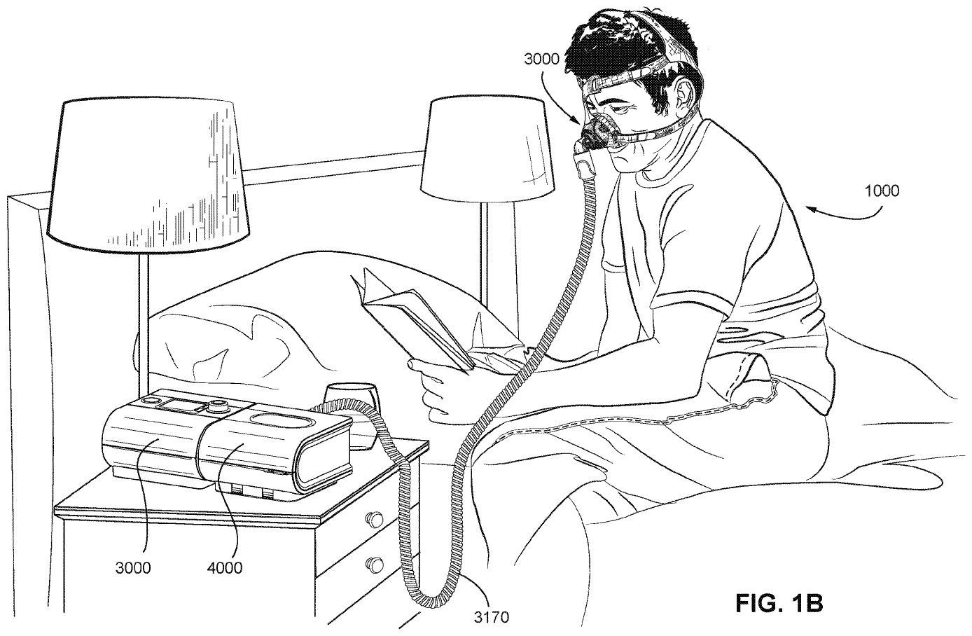

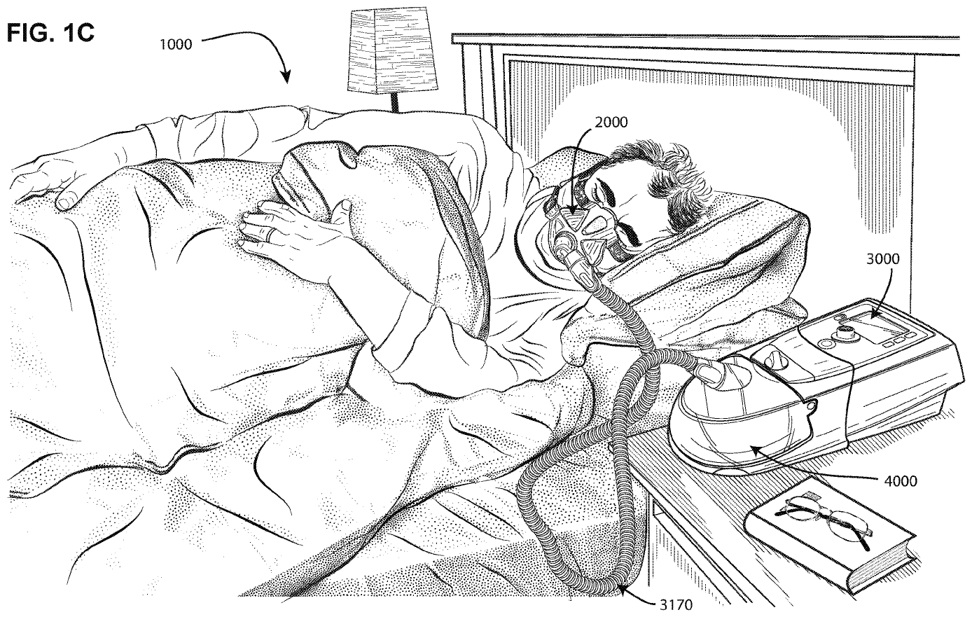

FIG. 1A shows a system including a patient 1000 wearing a patient interface 2000, in the form of nasal pillows, receiving a supply of air at positive pressure from an RPT device 3000. Air from the RPT device 3000 is humidified in a humidifier 4000, and passes along an air circuit 3170 to the patient 1000. A bed partner 1100 is also shown. The patient is sleeping in a supine sleeping position.

FIG. 1B shows a system including a patient 1000 wearing a patient interface 2000, in the form of a nasal mask, receiving a supply of air at positive pressure from an RPT device 3000. Air from the RPT device is humidified in a humidifier 4000, and passes along an air circuit 3170 to the patient 1000.

FIG. 1C shows a system including a patient 1000 wearing a patient interface 2000, in the form of a full-face mask, receiving a supply of air at positive pressure from an RPT device 3000. Air from the RPT device is humidified in a humidifier 4000, and passes along an air circuit 3170 to the patient 1000. The patient is sleeping in a side sleeping position.

### 3.2 Respiratory System and Facial Anatomy

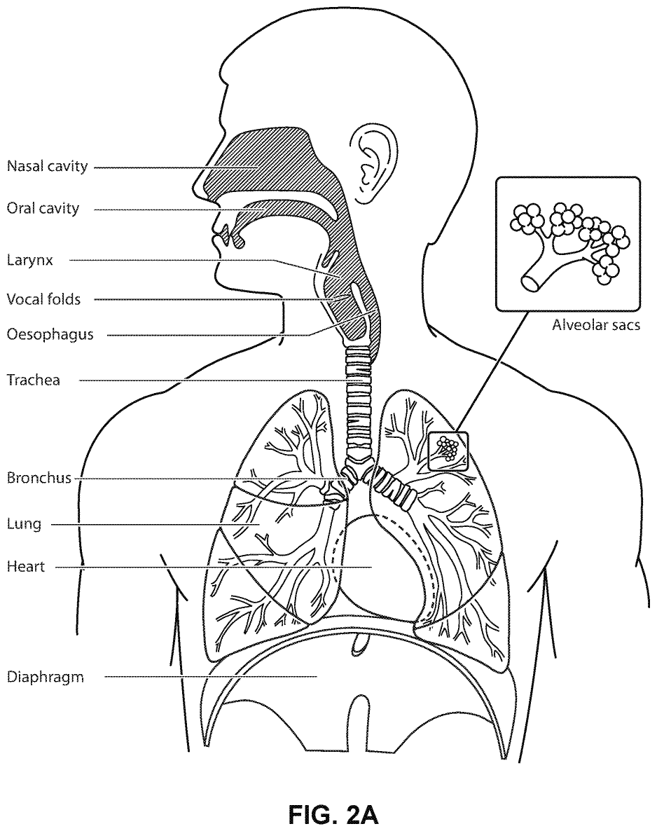

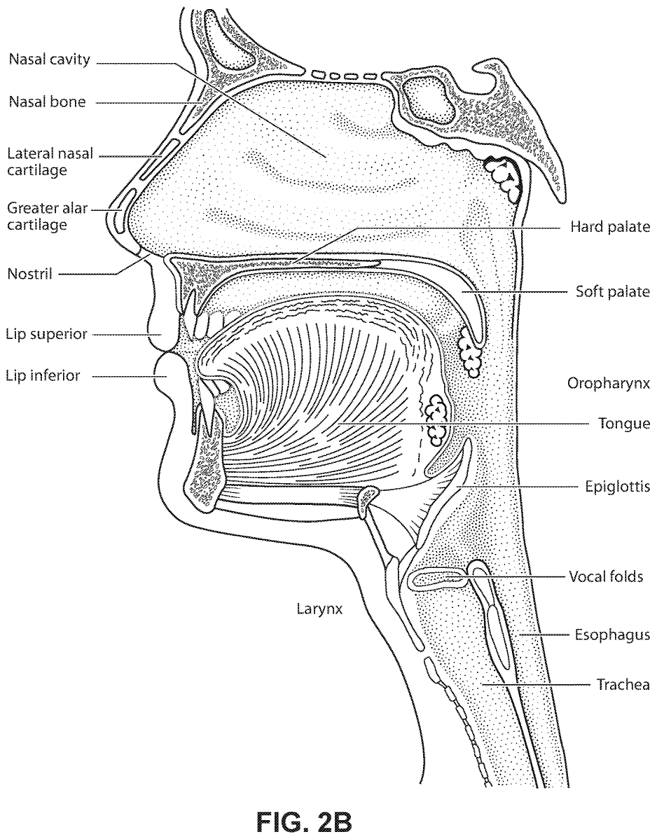

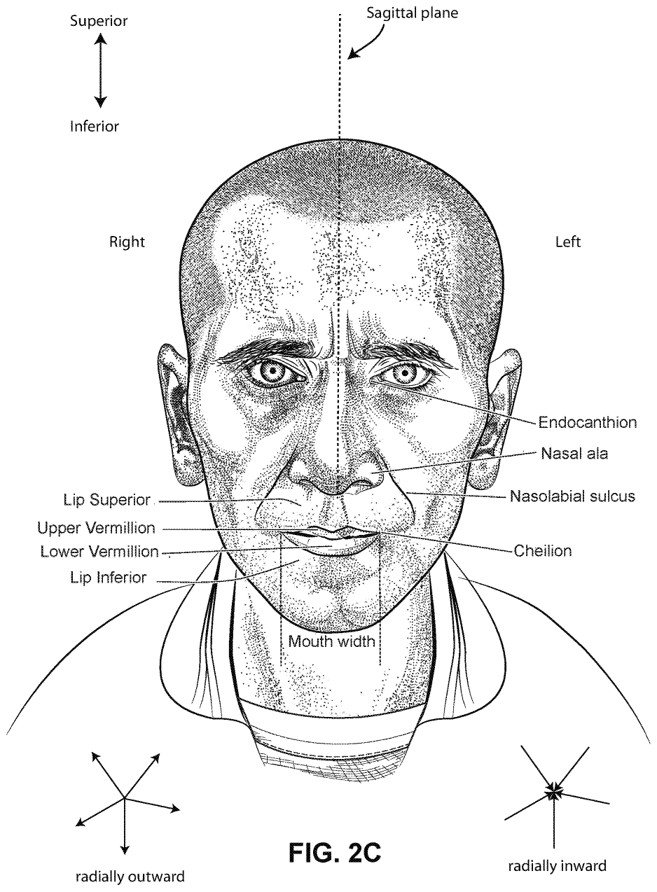

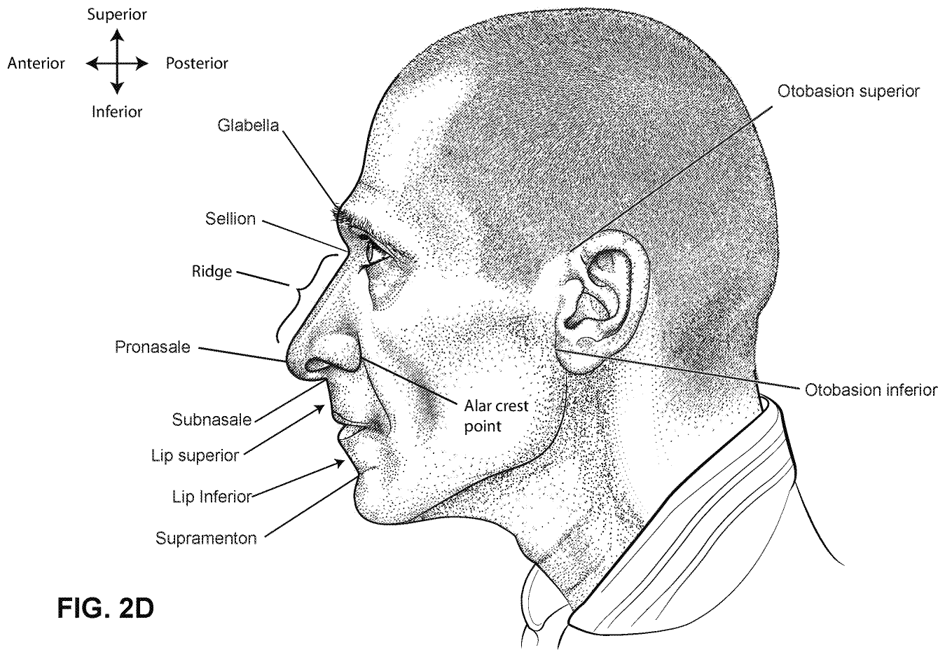

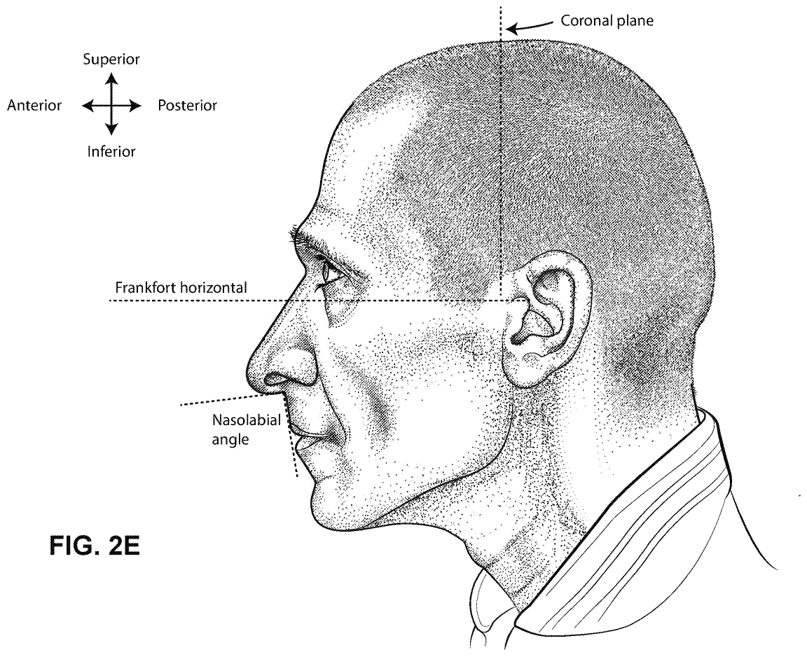

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

### 3.3 Patient Interface

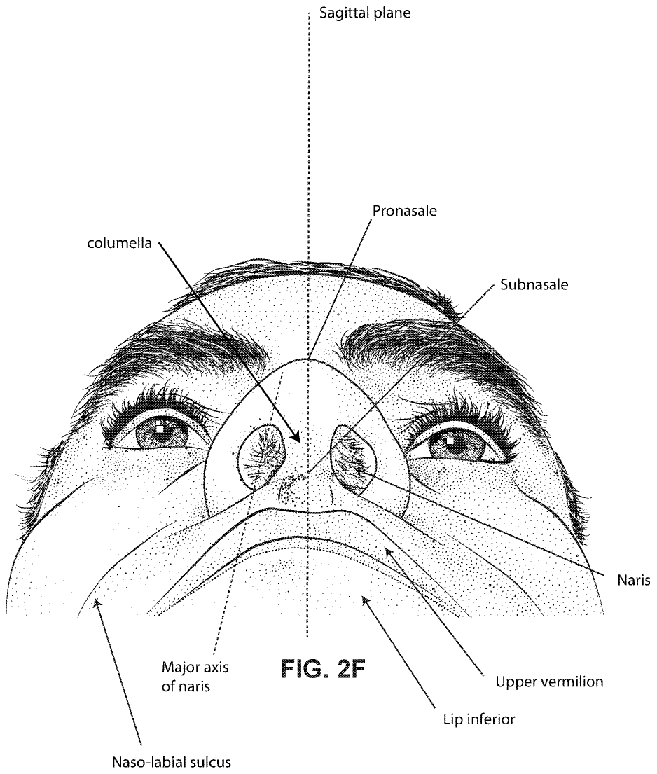

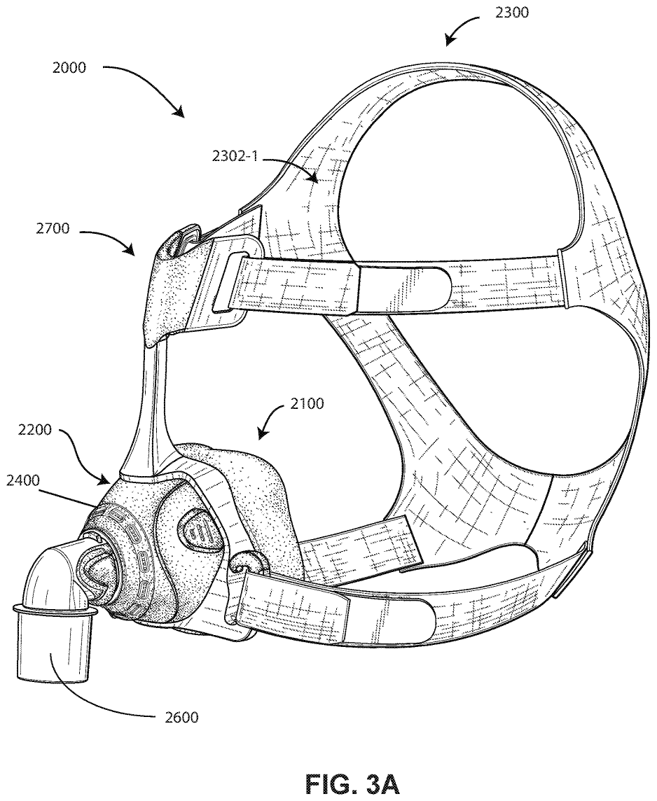

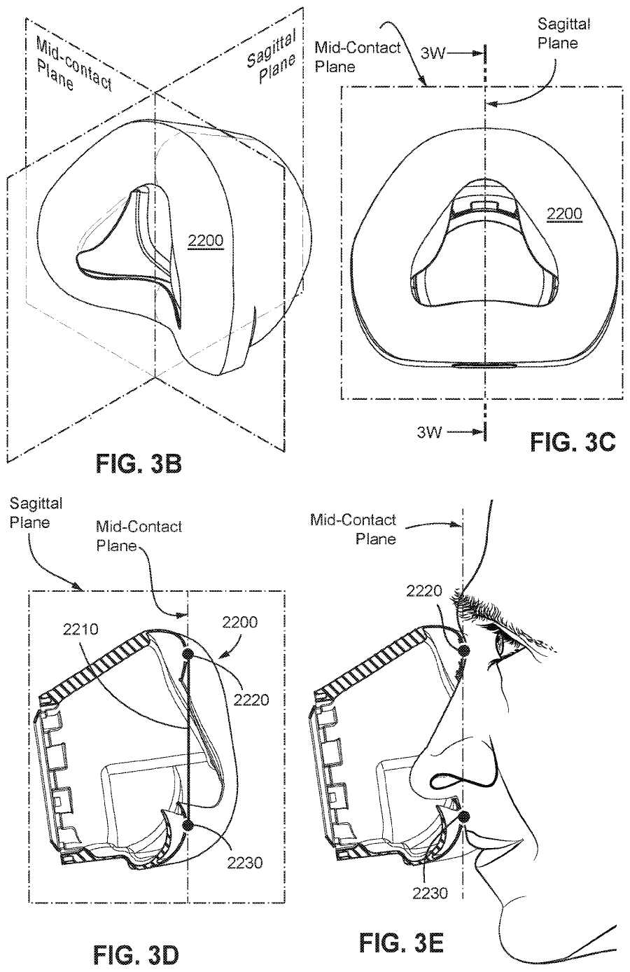

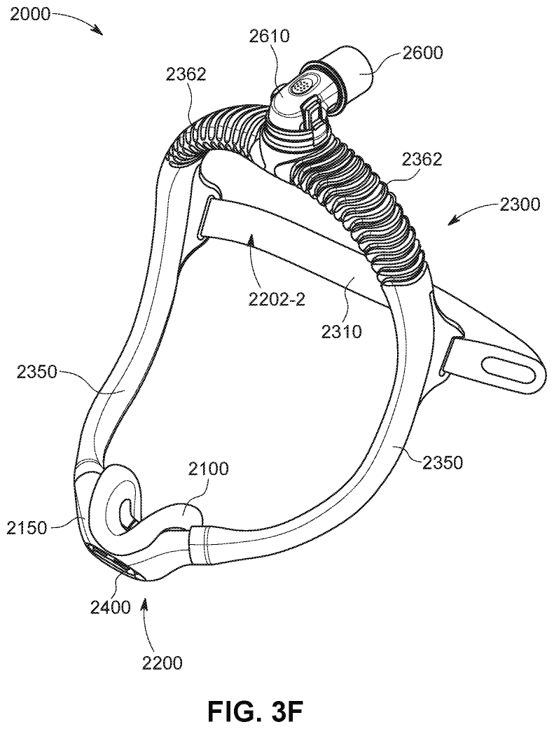

FIG. 3A shows a patient interface in the form of a nasal mask in accordance with one form of the present technology.

FIG. 3B shows a view of a plenum chamber 2200 showing a sagittal plane and a mid-contact plane.

FIG. 3C shows a view of a posterior of the plenum chamber of FIG. 3B. The direction of the view is normal to the mid-contact plane. The sagittal plane in FIG. 3D bisects the plenum chamber into left-hand and right-hand sides.

FIG. 3D shows a cross-section through the plenum chamber of FIG. 3C, the cross-section being taken at the sagittal plane shown in FIG. 3C. A ‘mid-contact’ plane is shown. The mid-contact plane is perpendicular to the sagittal plane. The orientation of the mid-contact plane corresponds to the orientation of a chord 2210 which lies on the sagittal plane and just touches the cushion of the plenum chamber at two points on the sagittal plane: a superior point 2220 and an inferior point 2230. Depending on the geometry of the cushion in this region, the mid-contact plane may be a tangent at both the superior and inferior points.

FIG. 3E shows the plenum chamber 2200 of FIG. 3B in position for use on a face. The sagittal plane of the plenum chamber 2200 generally coincides with the midsagittal plane of the face when the plenum chamber is in position for use. The mid-contact plane corresponds generally to the ‘plane of the face’ when the plenum chamber is in position for use. In FIG. 3E the plenum chamber 2200 is that of a nasal mask, and the superior point 2220 sits approximately on the sellion, while the inferior point 2230 sits on the lip superior.

FIG. 3F shows a patient interface having conduit headgear, in accordance with one form of the present technology.

### 3.4 Rpt Device

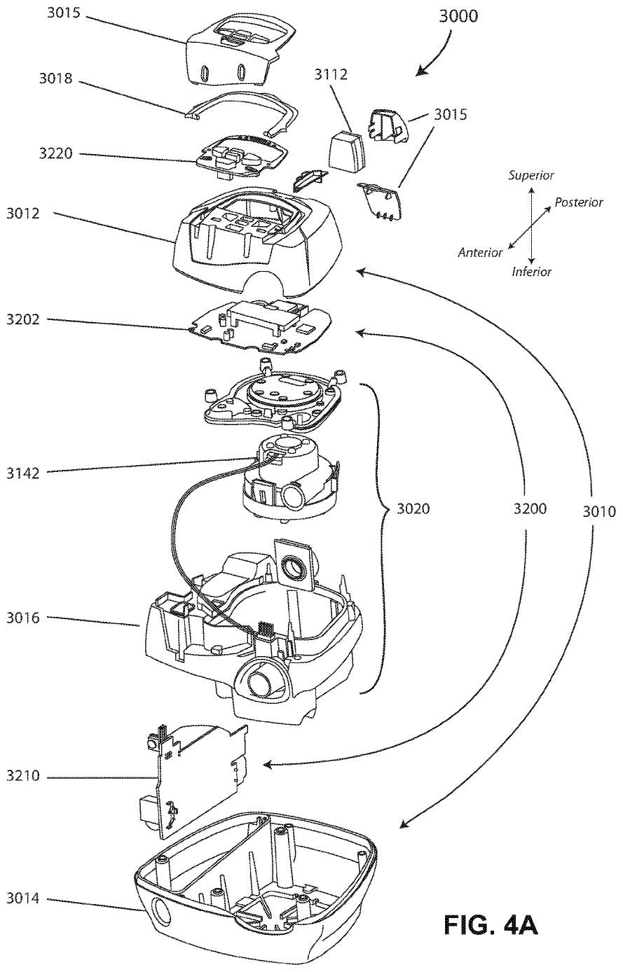

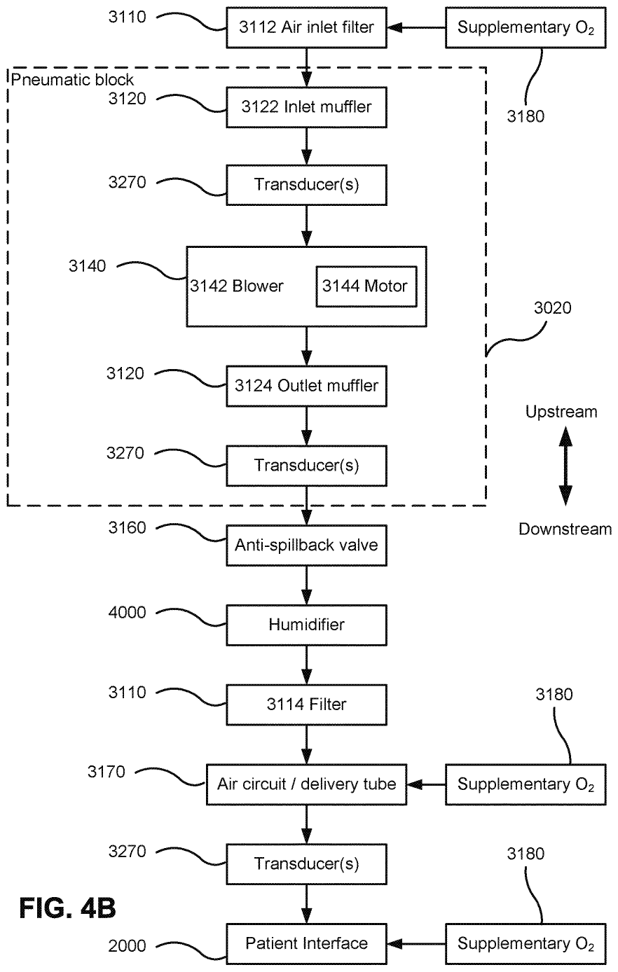

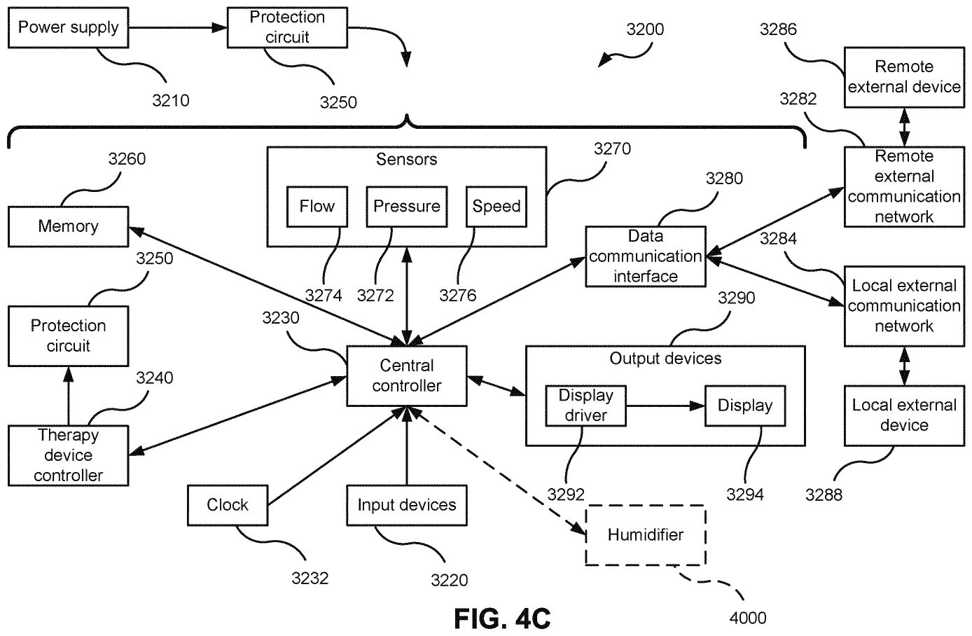

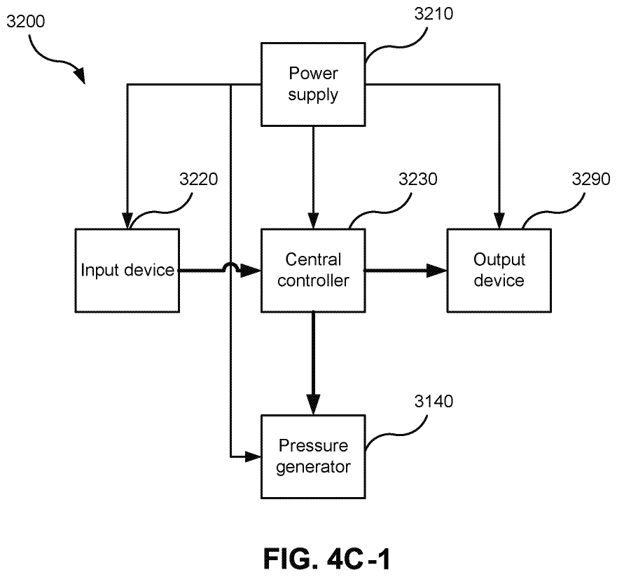

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4C-1 is a schematic diagram illustrating the interconnection of various electrical components of the RPT device.

### 3.5 Humidifier

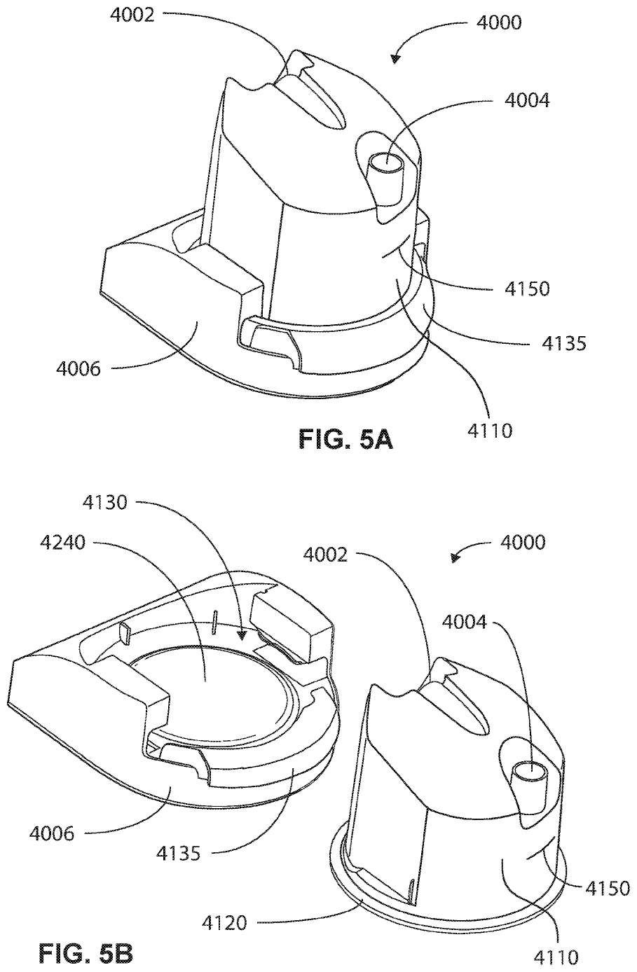

FIG. 5A shows an isometric view of a humidifier in accordance with one form of the present technology.

FIG. 5B shows an isometric view of a humidifier in accordance with one form of the present technology, showing a humidifier reservoir 4110 removed from the humidifier reservoir dock 4130.

### 3.6 Breathing Waveforms

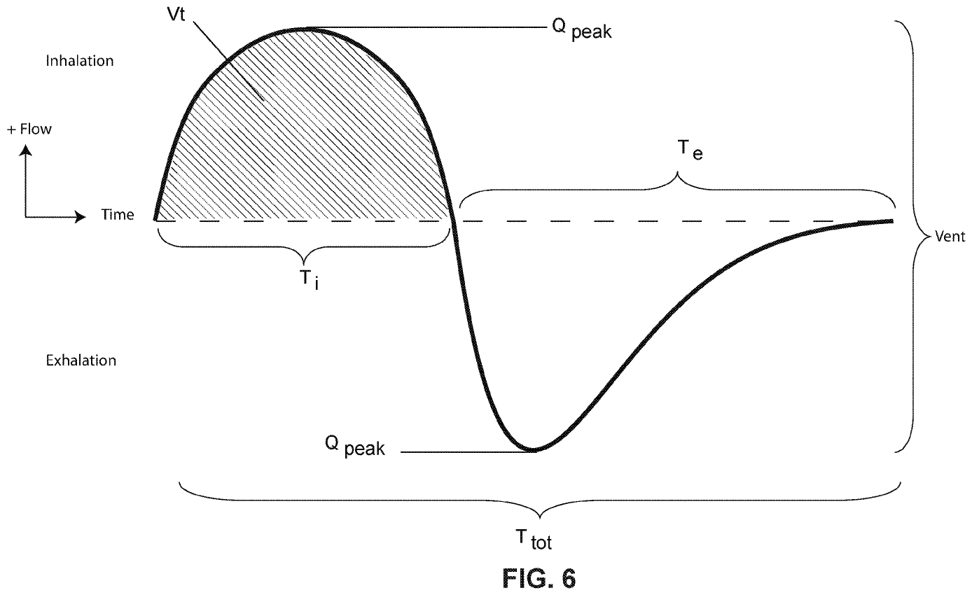

FIG. 6 shows a model typical breath waveform of a person while sleeping.

### 3.7 Patient Tracker

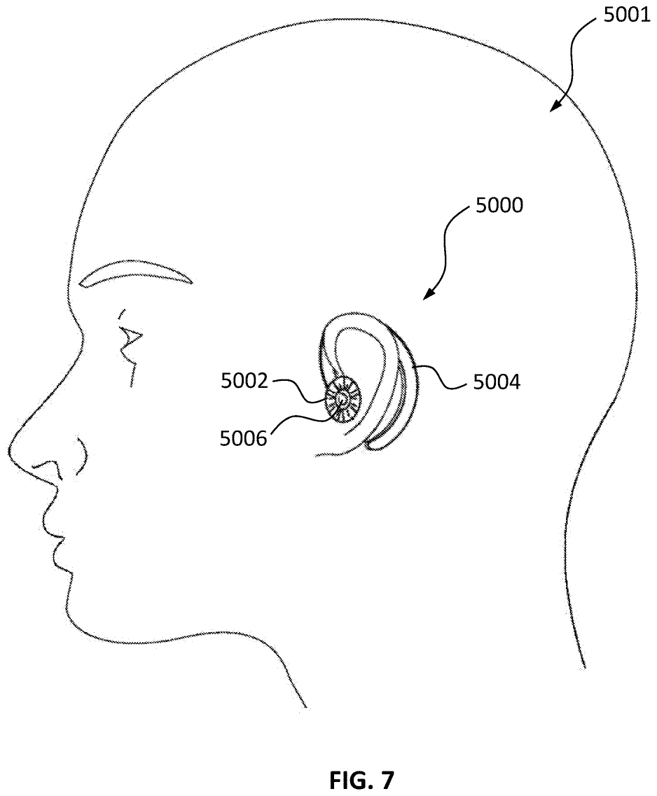

FIG. 7 shows a patient tracking device worn by a patient.

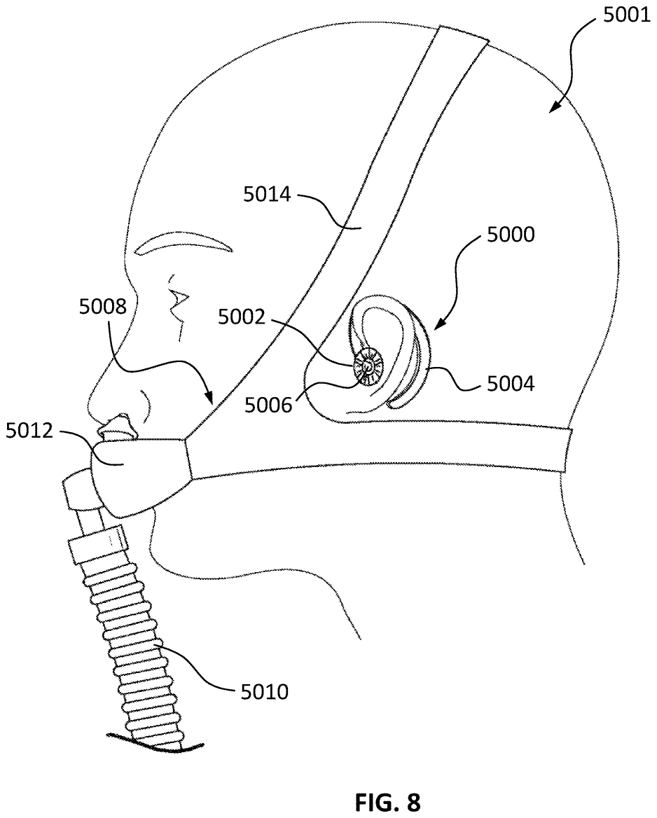

FIG. 8 shows the patient tracking device worn by the patient together with a patient interface.

### 3.8 Respiratory Therapy System in Conjunction with a Patient Tracker

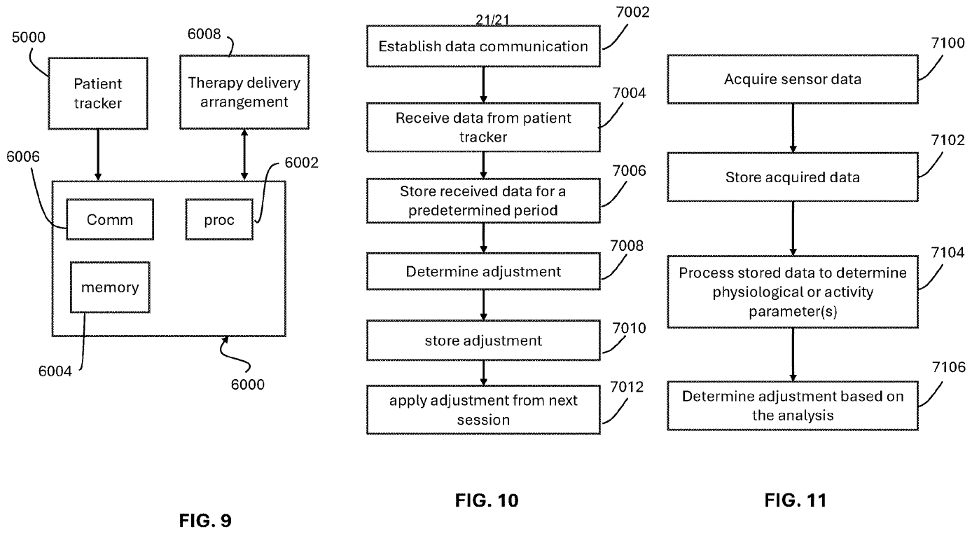

FIG. 9 shows a respiratory therapy system configured to receive data from a patient tracker.

FIG. 10 shows an example operation implemented by a therapy control program of the respiratory therapy system to determine therapy adjustment(s) based on data from the patient tracker.

FIG. 11 shows an example method implemented to utilise a patient tracker to determine therapy adjustments.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 3000 for supplying a flow of air to the patient 1000 via an air circuit 3170 and a patient interface 2000 or 2800.

### 4.3 Patient Interface

A non-invasive patient interface 2000, such as that shown in FIG. 3A, in accordance with one aspect of the present technology comprises the following functional aspects: a seal-forming structure 2100, a plenum chamber 2200, a positioning and stabilising structure 2300, a vent 2400, one form of connection port 2600 for connection to air circuit 3170, and a forehead support 2700. In some forms a functional aspect may be provided by one or more physical components. In some forms, one physical component may provide one or more functional aspects. In use the seal-forming structure 2100 is arranged to surround an entrance to the airways of the patient so as to maintain positive pressure at the entrance(s) to the airways of the patient 1000. The sealed patient interface 2000 is therefore suitable for delivery of positive pressure therapy.

The patient interface 2000 in accordance with one form of the present technology is constructed and arranged to be able to provide a supply of air at a positive pressure above the ambient, for example at least 2, 4, 6, 10, or 20 cmH2O with respect to ambient.

### 4.3.1 Seal-Forming Structure

In one form of the present technology, a seal-forming structure 2100 provides a target seal-forming region, and may additionally provide a cushioning function. The target seal-forming region is a region on the seal-forming structure 2100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 2100.

In certain forms of the present technology, the seal-forming structure 2100 is constructed from a biocompatible material, e.g. silicone rubber.

A seal-forming structure 2100 in accordance with the present technology may be constructed from a soft, flexible, resilient material such as silicone.

In certain forms of the present technology, a system is provided comprising more than one a seal-forming structure 2100, each being configured to correspond to a different size and/or shape range. For example the system may comprise one form of a seal-forming structure 2100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 2200 acting on its underside to urge it into tight sealing engagement with the face. The pressure assisted mechanism may act in conjunction with elastic tension in the positioning and stabilising structure.

In one form, the seal-forming structure 2100 comprises a sealing flange and a support flange. The sealing flange comprises a relatively thin member with a thickness of less than about 1 mm, for example about 0.25 mm to about 0.45 mm, which extends around the perimeter of the plenum chamber 2200. Support flange may be relatively thicker than the sealing flange. The support flange is disposed between the sealing flange and the marginal edge of the plenum chamber 2200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 2000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 2000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 2000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

4.3.1.6 Nasal pillows

In one form the seal-forming structure of the non-invasive patient interface 2000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nose-Only Masks

In one form, the patient interface 2000 comprises a seal-forming structure 2100 configured to seal around an entrance to the patient's nasal airways but not around the patient's mouth. The seal-forming structure 2100 may be configured to seal to the patient's lip superior. The patient interface 2000 may leave the patient's mouth uncovered. This patient interface 2000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 2100 configured to seal on the patient's face around the nose and over the bridge of the nose. A nasal mask may be generally triangular in shape. In one form, the non-invasive patient interface 2000 comprises a seal-forming structure 2100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 2000 shown in FIG. 1B has this type of seal-forming structure 2100. This patient interface 2000 may deliver a supply of air or breathable gas to both nares of patient 1000 through a single orifice.

Another form of nose-only mask may seal around an inferior periphery of the patient's nose without engaging the user's nasal ridge. This type of patient interface 2000 may be identified as a “nasal cradle” mask and the seal-forming structure 2100 may be identified as a “nasal cradle cushion”, for example. In one form, for example as shown in FIG. 3F, the seal-forming structure 2100 is configured to form a seal in use with inferior surfaces of the nose around the nares. The seal-forming structure 2100 may be configured to seal around the patient's nares at an inferior periphery of the patient's nose including to an inferior and/or anterior surface of a pronasale region of the patient's nose and to the patient's nasal alae. The seal-forming structure 2100 may seal to the patient's lip superior. The shape of the seal-forming structure 2100 may be configured to match or closely follow the underside of the patient's nose and may not contact a nasal bridge region of the patient's nose or any portion of the patient's nose superior to the pronasale. In one form of nasal cradle cushion, the seal-forming structure 2100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 2100 may comprise a single opening to provide a flow or air or breathable gas to both of the patient's nares.

In some forms, a nose-only mask may comprise nasal pillows, described above.

### 4.3.1.8 Nose and Mouth Masks

In one form, the patient interface 2000 comprises a seal-forming structure 2100 configured to seal around an entrance to the patient's nasal airways and also around the patient's mouth. The seal-forming structure 2100 may be configured to seal to the patient's face proximate a chin region. This patient interface 2000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 2100 configured to seal on the patient's face around the nose, below the mouth and over the bridge of the nose. A nose-and-mouth mask may be generally triangular in shape. In one form the patient interface 2000 comprises a seal-forming structure 2100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 2000 shown in FIG. 1C is of this type. This patient interface 2000 may deliver a supply of air or breathable gas to both nares and mouth of patient 1000 through a single orifice. This type of seal-forming structure 2100 may be referred to as a “nose-and-mouth cushion”.

In another form the patient interface 2000 comprises a seal-forming structure 2100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 2100 may also form a seal against a patient's lip superior. A patient interface 2000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 2000 may have a nasal portion and an oral portion, the nasal portion sealing to the patient's face at similar locations to a nasal cradle mask.

In a further form of nose and mouth mask, the patient interface 2000 may comprise a seal-forming structure 2100 having a nasal portion comprising nasal pillows and an oral portion configured to form a seal to the patient's face around the patient's mouth.

In some forms, the seal-forming structure 2100 may have a nasal portion that is separate and distinct from an oral portion. In other forms, a seal-forming structure 2100 may form a contiguous seal around the patient's nose and mouth.

It is to be understood that the above examples of different forms of patient interface 2000 do not constitute an exhaustive list of possible configurations. In some forms a patient interface 2000 may comprise a combination of different features of the above described examples of nose-only and nose and mouth masks.

### 4.3.2 Plenum Chamber

The plenum chamber 2200 has a perimeter that is shaped to be complementary to the surface contour of the face of an average person in the region where a seal will form in use. In use, a marginal edge of the plenum chamber 2200 is positioned in close proximity to an adjacent surface of the face. Actual contact with the face is provided by the seal-forming structure 2100. The seal-forming structure 2100 may extend in use about the entire perimeter of the plenum chamber 2200. In some forms, the plenum chamber 2200 and the seal-forming structure 2100 are formed from a single homogeneous piece of material.

In certain forms of the present technology, the plenum chamber 2200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 2200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 2200 is constructed from a translucent material. The use of a translucent material can reduce the obtrusiveness of the patient interface, and help improve compliance with therapy.

In some forms, the plenum chamber 2200 is constructed from a rigid material such as polycarbonate. The rigid material may provide support to the seal-forming structure.

In some forms, the plenum chamber 2200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 2200 may be made from a material having Young's modulus of 0.1 GPa or lower, for example rubber. In other forms of the technology the plenum chamber 2200 may be made from a material having a Young's modulus of 0.7 MPa or less, for example between 0.7 MPa and 0.3 MPa. An example of such a material is silicone.

### 4.3.3 Positioning and Stabilising Structure

The seal-forming structure 2100 of the patient interface 2000 of the present technology may be held in sealing position in use by the positioning and stabilising structure 2300. The positioning and stabilising structure 2300 may comprise and function as “headgear” since it engages the patient's head in order to hold the patient interface 2000 in a sealing position. Examples of a positioning and stabilising structure may be shown in FIG. 3A.

In one form the positioning and stabilising structure 2300 provides a retention force at least sufficient to overcome the effect of the positive pressure in the plenum chamber 2200 to lift off the face (i.e., Fₚₗₑₙᵤₘ).

In one form the positioning and stabilising structure 2300 provides a retention force to overcome the effect of the gravitational force on the patient interface 2000.

In one form of the present technology, a positioning and stabilising structure 2300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 2300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 2300 comprises a strap that is bendable and e.g. non-rigid. An advantage of this aspect is that the strap is more comfortable for a patient to lie upon while the patient is sleeping.

In certain forms of the present technology, a positioning and stabilising structure 2300 comprises a strap constructed to be breathable to allow moisture vapour to be transmitted through the strap,

In certain forms of the present technology, a system is provided comprising more than one positioning and stabilising structure 2300, each being configured to provide a retaining force to correspond to a different size and/or shape range. For example the system may comprise one form of positioning and stabilising structure 2300 suitable for a large sized head, but not a small sized head, and another. suitable for a small sized head, but not a large sized head.

### 4.3.3.1 Conduit Headgear

### 4.3.3.1.1 Conduit Headgear Tubes

In some forms of the present technology, the positioning and stabilising structure 2300 comprises one or more headgear tubes 2350 that deliver pressurised air received from a conduit forming part of the air circuit 3170 from the RPT device to the patient's airways, for example through the plenum chamber 2200 and seal-forming structure 2100. In the form of the present technology illustrated in FIG. 3F, the positioning and stabilising structure 2300 comprises two tubes 2350 that deliver air to the plenum chamber 2200 from the air circuit 3170. The tubes 2350 are configured to position and stabilise the seal-forming structure 2100 of the patient interface 2000 at the appropriate part of the patient's face (for example, the nose and/or mouth) in use. This allows the conduit of air circuit 3170 providing the flow of pressurised air to connect to a connection port 2600 of the patient interface in a position other than in front of the patient's face, for example on top of the patient's head.

In the form of the present technology illustrated in FIG. 3F, the positioning and stabilising structure 2300 comprises two tubes 2350, each tube 2350 being positioned in use on a different side of the patient's head and extending across the respective cheek region, above the respective ear (superior to the otobasion superior on the patient's head) to the elbow 2610 on top of the head of the patient 1000. This form of technology may be advantageous because, if a patient sleeps with their head on its side and one of the tubes 2350 is compressed to block or partially block the flow of gas along the tube 2350, the other tube 2350 remains open to supply pressurised gas to the patient. In other examples of the technology, the patient interface 2000 may comprise a different number of tubes, for example one tube, or two or more tubes.

In one example in which the patient interface has one tube 2350, the single tube 2350 is positioned on one side of the patient's head in use (e.g. across one cheek region) and a strap forms part of the positioning and stabilising structure 2300 and is positioned on the other side of the patient's head in use (e.g. across the other region) to assist in securing the patient interface 2000 on the patient's head. For example, the tube 2350 and the strap may each be under tension in use in order to assist in maintaining the seal-forming structure 2100 in a sealing position.

In one form, the tube 2350 may be at least partially extensible so that the tube 2350 and the strap may adjust substantially equal lengths when worn by a patient. This may allow for substantially symmetrical adjustments between the tube 2350 and the strap so that the seal-forming structure remains substantially in the middle.

In the form of the technology shown in FIG. 3F, the two tubes 2350 are fluidly connected at superior ends to each other and to the connection port 2600. In some examples, the two tubes 2350 are integrally formed while in other examples the tubes 2350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 2350. Additionally, the T-shaped connector may have a third arm or opening providing the connection port 2600 for fluid connection to the air circuit 3170 in use. The opening may be an inlet 2332 (see e.g., 7C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 2300.

In some forms, at least one of the arms of the T-shaped connector (or Y-shaped connector) may be at least partially rigidised. This may assist in maintaining the shape of the connector so that bending of the connector does not close the airflow path.

The tubes 2350 may be formed from a flexible material, such as an elastomer, e.g. silicone or TPE, and/or from one or more textile and/or foam materials. The tubes 2350 may have a preformed shape and may be able to be bent or moved into another shape upon application of a force but may return to the original preformed shape in the absence of said force. The tubes 2350 may be generally arcuate or curved in a shape approximating the contours of a patient's head between the top of the head and the nasal or oral region.

In some examples, the one or more tubes 2350 are crush resistant to resist being blocked if crushed during use, for example if squashed between a patient's head and pillow, especially if there is only one tube 2350. The tubes 2350 may be formed with a sufficient structural stiffness to resist crushing or may be as described in U.S. Pat. No. 6,044,844, the contents of which are incorporated herein by reference.

Each tube 2350 may be configured to receive a flow of air from the connection port 2600 on top of the patient's head and to deliver the flow of air to the seal-forming structure 2100 at the entrance of the patient's airways. In the example shown in FIG. 3F, each tube 2350 lies in use on a path extending from the plenum chamber 2200 across the patient's cheek region and superior to the patient's ear to the elbow 2610. For example, a portion of each tube 2350 proximate the plenum chamber 2200 may overlie a maxilla region of the patient's head in use. Another portion of each tube 2350 may overlie a region of the patient's head superior to an otobasion superior of the patient's head. Each of the tubes 2350 may also lie over the patient's sphenoid bone and/or temporal bone and either or both of the patient's frontal bone and parietal bone. The elbow 2610 may be located in use over the patient's parietal bone, over the frontal bone and/or over the junction therebetween (e.g. the coronal suture).

In certain forms of the present technology the patient interface 2000 is configured such that the connection port 2600 can be positioned in a range of positions across the top of the patient's head so that the patient interface 2000 can be positioned as appropriate for the comfort or fit of an individual patient. In some examples, the headgear tubes 2350 are configured to allow movement of an upper portion of the patient interface 2000 (e.g. a connection port 2600) with respect to a lower portion of the patient interface 2000 (e.g. a plenum chamber 2200). That is, the connection port 2600 may be at least partially decoupled from the plenum chamber 2200. In this way, the seal-forming structure 2100 may form an effective seal with the patient's face irrespective of the position of the connection port 2600 (at least within a predetermined range of positions) on the patient's head.

As described above, in some examples of the present technology the patient interface 2000 comprises a seal-forming structure 2100 in the form of a cradle cushion which lies generally under the nose and seals to an inferior periphery of the nose (e.g. an under-the-nose cushion). The positioning and stabilising structure 2300, including the tubes 2350 may be structured and arranged to pull the seal-forming structure 2100 into the patient's face under the nose with a sealing force in a posterior and superior direction (e.g. a posterosuperior direction). A sealing force with a posterosuperior direction may cause the seal-forming structure 2100 to form a good seal to both the inferior periphery of the patient's nose and anterior-facing surfaces of the patient's face, for example on either side of the patient's nose and the patient's lip superior.

### 4.3.3.1.2 Conduit Headgear Connection Port

In certain forms of the present technology, the patient interface 2000 may comprise a connection port 2600 located proximal to a superior, lateral or posterior portion of a patient's head. For example, in the form of the present technology illustrated in FIG. 3F, the connection port 2600 is located on top of the patient's head (e.g. at a superior location with respect to the patient's head). In this example the patient interface 2000 comprises an elbow 2610 forming the connection port 2600. The elbow 2610 may be configured to fluidly connect with a conduit of an air circuit 3170. The elbow 2610 may be configured to swivel with respect to the positioning and stabilising structure 2300 to at least partially decouple the conduit from the positioning and stabilising structure 2300. In some examples the elbow 2610 may be configured to swivel by rotation about a substantially vertical axis and, in some particular examples, by rotation about two or more axes. In some examples the elbow may comprise or be connected to the tubes 2350 by a ball-and-socket joint. The connection portion 2600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 4.3.3.1.3 Headgear Tube Fluid Connections

The two tubes 2350 are fluidly connected at their inferior ends to the plenum chamber 2200. In certain forms of the technology, the connection between the tubes 2350 and the plenum chamber 2200 is achieved by connection of two rigid connectors. The tubes 2350 and plenum chamber 2200 may be configured to enable the patient to easily connect the two components together in a reliable manner. The tubes 2350 and plenum chamber 2200 may be configured to provide tactile and/or audible feedback in the form of a ‘re-assuring click’ or a similar sound, so that the patient may easily know that each tube 2350 has been correctly connected to the plenum chamber 2200. In one form, the tubes 2350 are formed from a silicone or textile material and the inferior end of each of the silicone tubes 2350 is overmolded to a rigid connector made, for example, from polypropylene, polycarbonate, nylon or the like. The rigid connector on each tube 2350 may comprise a female mating feature configured to connect with a male mating feature on the plenum chamber 2200. Alternatively, the rigid connector on each tube 2350 may comprise a male mating feature configured to connect to a female mating feature on the plenum chamber 2200. In other examples the tubes 2350 may each comprise a male or female connector formed from a flexible material, such as silicone or TPE, for example the same material from which the tubes 2350 are formed.

In other examples a compression seal is used to connect each tube 2350 to the plenum chamber 2200. For example, a resiliently flexible (e.g. silicone) tube 2350 without a rigid connector may be configured to be squeezed to reduce its diameter so that it can be compressed into a port in the plenum chamber 2200 and the inherent resilience of the silicone pushes the tube 2350 outwards to seal the tube 2350 in the port in an air-tight manner. Alternatively, in a hard-to-hard type engagement between the tube 2350 and the plenum chamber 2200, each tube 2350 and/or plenum chamber 2200 may comprise a pressure activated seal, for example a peripheral sealing flange. When pressurised gas is supplied through the tubes 2350 the sealing flange may be urged against the join between the tubes and a circumferential surface around a port or connector of the plenum chamber 2200 to form or enhance a seal between the tube 2350 and plenum chamber 2200.

### 4.3.3.2 Headgear Straps

In some forms, the positioning and stabilising structure 2300 may include headgear 2302 with at least one strap which may be worn by the patient in order to assist in properly orienting the seal-forming structure 2100 against the patient's face (e.g., in order to limit or prevent leaks).

As described above, some forms of the headgear 2302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 2302.

In certain forms, the headgear 2302 may be at least partially extensible. For example, the headgear 2302 may include elastic, or a similar extensible material. For example, the entire headgear 2302 may be extensible or selected portions may be extensible (or more extensible than surrounding portions). This may allow the headgear 2302 to stretch while under tension, which may assist in providing a sealing force for the seal-forming structure 2100.

Two forms of the headgear, four-point headgear 2302-1 (see FIG. 3A) and two-point headgear 2302-2 (see FIG. 3F).

### 4.3.4 Vent

In one form, the patient interface 2000 includes a vent 2400 constructed and arranged to allow for the washout of exhaled gases, e.g. carbon dioxide.

In certain forms the vent 2400 is configured to allow a continuous vent flow from an interior of the plenum chamber 2200 to ambient whilst the pressure within the plenum chamber is positive with respect to ambient. The vent 2400 is configured such that the vent flow rate has a magnitude sufficient to reduce rebreathing of exhaled CO2 by the patient while maintaining the therapeutic pressure in the plenum chamber in use.

One form of vent 2400 in accordance with the present technology comprises a plurality of holes, for example, about 20 to about 80 holes, or about 40 to about 60 holes, or about 45 to about 55 holes.

The vent 2400 may be located in the plenum chamber 2200. Alternatively, the vent 2400 is located in a decoupling structure, e.g., a swivel.

### 4.3.5 Decoupling Structure(s)

In one form the patient interface 2000 includes at least one decoupling structure, for example, a swivel or a ball and socket.

### 4.3.6 Connection Port

Connection port 2600 allows for connection to the air circuit 3170.

### 4.3.7 Forehead Support

In one form, the patient interface 2000 includes a forehead support 2700.

### 4.3.8 Anti-Asphyxia Valve

In one form, the patient interface 2000 includes an anti-asphyxia valve.

### 4.3.9 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 2350 (see e.g., FIG. 3F). This may be referred to as a “tube up” configuration and may position a connection port at the top of the patient's head. In other example, the patient may receive the flow of pressurized air through a conduit connected to the plenum chamber 2200, for example through the connection port 2600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 4.4 RPT Device

An RPT device 3000 in accordance with one aspect of the present technology comprises mechanical, pneumatic, and/or electrical components and is configured to execute one or more algorithms 3300, such as any of the methods, in whole or in part, described herein. The RPT device 3000 may be configured to generate a flow of air for delivery to a patient's airways, such as to treat one or more of the respiratory conditions described elsewhere in the present document.

The RPT device may have an external housing 3010, formed in two parts, an upper portion 3012 and a lower portion 3014. Furthermore, the external housing 3010 may include one or more panel(s) 3015. The RPT device 3000 comprises a chassis 3016 that supports one or more internal components of the RPT device 3000. The RPT device 3000 may include a handle 3018.

The pneumatic path of the RPT device 3000 may comprise one or more air path items, e.g., an inlet air filter 3112, an inlet muffler 3122, a pressure generator 3140 capable of supplying air at positive pressure (e.g., a blower 3142), an outlet muffler 3124 and one or more transducers 3270, such as pressure sensors 3272 and flow rate sensors 3274.

One or more of the air path items may be located within a removable unitary structure which will be referred to as a pneumatic block 3020. The pneumatic block 3020 may be located within the external housing 3010. In one form a pneumatic block 3020 is supported by, or formed as part of the chassis 3016.

As shown in FIG. 4C, the RPT device 3000 may have an electrical power supply 3210, one or more input devices 3220, a central controller 3230, a therapy device controller 3240, a pressure generator 3140, one or more protection circuits 3250, memory 3260, transducers 3270, data communication interface 3280 and one or more output devices 3290. Electrical components 3200 may be mounted on a single Printed Circuit Board Assembly (PCBA) 3202. In an alternative form, the RPT device 3000 may include more than one PCBA 3202.

### 4.4.1 RPT Device Mechanical & Pneumatic Components

An RPT device may comprise one or more of the following components in an integral unit. In an alternative form, one or more of the following components may be located as respective separate units.

### 4.4.1.1 Air Filter(s)

An RPT device in accordance with one form of the present technology may include an air filter 3110, or a plurality of air filters 3110.

In one form illustrated in FIG. 4B, an inlet air filter 3112 is located at the beginning of the pneumatic path upstream of a pressure generator 3140.

In one form illustrated in FIG. 4B, an outlet air filter 3114, for example an antibacterial filter, is located between an outlet of the pneumatic block 3020 and a patient interface 2000 or 2800.

### 4.4.1.2 Muffler(s)

An RPT device in accordance with one form of the present technology may include a muffler 3120, or a plurality of mufflers 3120.

In one form of the present technology (see e.g., FIG. 4B), an inlet muffler 3122 is located in the pneumatic path upstream of a pressure generator 3140.

In one form of the present technology, an outlet muffler 3124 is located in the pneumatic path between the pressure generator 3140 and a patient interface 2000 or 2800.

### 4.4.1.3 Pressure Generator

In one form of the present technology, a pressure generator 3140 for producing a flow, or a supply, of air at positive pressure is a controllable blower 3142. For example, the blower 3142 may include a brushless DC motor 3144 with one or more impellers. The impellers may be located in a volute. The blower may be capable of delivering a supply of air, for example at a rate of up to about 120 litres/minute, at a positive pressure in a range from about 4 cmH20 to about 20 cmH2O, or in other forms up to about 30 cmH20 when delivering respiratory pressure therapy. The blower may be as described in any one of the following patents or patent applications the contents of which are incorporated herein by reference in their entirety: U.S.

U.S. Pat. Nos. 7,866,944; 8,638,014; 8,636,479; and PCT Patent Application Publication No. WO 2013/020167.

The pressure generator 3140 may be under the control of the therapy device controller 3240.

In other forms, a pressure generator 3140 may be a piston-driven pump, a pressure regulator connected to a high pressure source (e.g. compressed air reservoir), or a bellows.

### 4.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 4B), one or more transducers 3270 are located upstream and/or downstream of the pressure generator 3140. The one or more transducers 3270 may be constructed and arranged to generate signals representing properties of the flow of air such as a flow rate, a pressure or a temperature at that point in the pneumatic path.

In one form of the present technology, one or more transducers 3270 may be located proximate to the patient interface 2000 or 2800.

In one form, a signal from a transducer 3270 may be filtered, such as by low-pass, high-pass or band-pass filtering.

### 4.4.1.4.1 Flow Rate Sensor

A flow rate sensor 3274 in accordance with the present technology may be based on a differential pressure transducer, for example, an SDP600 Series differential pressure transducer from SENSIRION.

In one form, a signal generated by the flow rate sensor 3274 and representing a flow rate is received by the central controller 3230.

### 4.4.1.4.2 Pressure Sensor

A pressure sensor 3272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 3272 and representing a pressure is received by the central controller 3230.

### 4.4.1.4.3 Motor Speed Transducer

In one form of the present technology a motor speed transducer 3276 is used to determine a rotational velocity of the motor 3144 and/or the blower 3142. A motor speed signal from the motor speed transducer 3276 may be provided to the therapy device controller 3240. The motor speed transducer 3276 may, for example, be a speed sensor, such as a Hall effect sensor.

### 4.4.1.5 Anti-Spill Back Valve

As shown in FIG. 4B, one form of the present technology, an anti-spill back valve 3160 is located between the humidifier 4000 and the pneumatic block 3020. The anti-spill back valve is constructed and arranged to reduce the risk that water will flow upstream from the humidifier 4000, for example to the motor 3144.

### 4.4.2 RPT Device Electrical Components

### 4.4.2.1 Power Supply

A power supply 3210 may be located internal or external of the external housing 3010 of the RPT device 3000.

In one form of the present technology, power supply 3210 provides electrical power to the RPT device 3000 only. In another form of the present technology, power supply 3210 provides electrical power to both RPT device 3000 and humidifier 4000.

As illustrated in FIG. 4C-1, the power supply 3210 may provide electrical power to the input device 3220, the central controller 3230, the output device 3290, and the pressure generator 3140. The power supply 3210 may also provide electric energy to other components of the RPT device 3000 (or the humidifier 4000, as described above).

### 4.4.2.2 Input Devices

In one form of the present technology, an RPT device 3000 includes one or more input devices 3220 in the form of buttons, switches or dials to allow a person to interact with the device. The buttons, switches or dials may be physical devices, or software devices accessible via a touch screen. The buttons, switches or dials may, in one form, be physically connected to the external housing 3010, or may, in another form, be in wireless communication with a receiver that is in electrical connection to the central controller 3230.

In one form, the input device 3220 may be constructed and arranged to allow a person to select a value and/or a menu option.

### 4.4.2.3 Central Controller

In one form of the present technology, the central controller 3230 is one or a plurality of processors suitable to control an RPT device 3000. The central controller 3230 is show in FIGS. 4C and 4C-1.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 3230 is a dedicated electronic circuit.

In one form, the central controller 3230 is an application-specific integrated circuit. In another form, the central controller 3230 comprises discrete electronic components.

The central controller 3230 may be configured to receive input signal(s) from one or more transducers 3270, one or more input devices 3220, and/or the humidifier 4000.

The central controller 3230 may be configured to provide output signal(s) to one or more of an output device 3290, a pressure generator 3140, a therapy device controller 3240, a data communication interface 3280, and/or the humidifier 4000.

In some forms of the present technology, the central controller 3230 is configured to implement the one or more methodologies described herein, such as the one or more algorithms 3300 which may be implemented with processor-control instructions, expressed as computer programs stored in a non-transitory computer readable storage medium, such as memory 3260. In some forms of the present technology, the central controller 3230 may be integrated with an RPT device 3000. However, in some forms of the present technology, some methodologies may be performed by a remotely located device. For example, the remotely located device may determine control settings for a ventilator or detect respiratory related events by analysis of stored data such as from any of the sensors described herein.

### 4.4.2.4 Clock

The RPT device 3000 may include a clock 3232 that is connected to the central controller 3230.

### 4.4.2.5 Therapy Device Controller

In one form of the present technology, therapy device controller 3240 is a therapy control module 3330 that forms part of the algorithms 3300 executed by the central controller 3230.

In one form of the present technology, therapy device controller 3240 is a dedicated motor control integrated circuit. For example, in one form a MC33035 brushless DC motor controller, manufactured by ONSEMI is used.

### 4.4.2.6 Protection Circuits

The one or more protection circuits 3250 in accordance with the present technology may comprise an electrical protection circuit, a temperature and/or pressure safety circuit.

### 4.4.2.7 Memory

In accordance with one form of the present technology the RPT device 3000 includes memory 3260, e.g., non-volatile memory. In some forms, memory 3260 may include battery powered static RAM. In some forms, memory 3260 may include volatile RAM.

Memory 3260 may be located on the PCBA 3202. Memory 3260 may be in the form of EEPROM, or NAND flash.

Additionally, or alternatively, RPT device 3000 includes a removable form of memory 3260, for example a memory card made in accordance with the Secure Digital (SD) standard.

In one form of the present technology, the memory 3260 acts as a non-transitory computer readable storage medium on which is stored computer program instructions expressing the one or more methodologies described herein, such as the one or more algorithms 3300.

### 4.4.2.8 Data Communication Systems

In one form of the present technology, a data communication interface 3280 is provided, and is connected to the central controller 3230 (see e.g., FIG. 4C). Data communication interface 3280 may be connectable to a remote external communication network 3282 and/or a local external communication network 3284. The remote external communication network 3282 may be connectable to a remote external device 3286. The local external communication network 3284 may be connectable to a local external device 3288.

In one form, data communication interface 3280 is part of the central controller 3230. In another form, data communication interface 3280 is separate from the central controller 3230, and may comprise an integrated circuit or a processor.

In one form, remote external communication network 3282 is the Internet. The data communication interface 3280 may use wired communication (e.g. via Ethernet, or optical fibre) or a wireless protocol (e.g. CDMA, GSM, LTE) to connect to the Internet.

In one form, local external communication network 3284 utilises one or more communication standards, such as Bluetooth, or a consumer infrared protocol.

In one form, remote external device 3286 is one or more computers, for example a cluster of networked computers. In one form, remote external device 3286 may be virtual computers, rather than physical computers. In either case, such a remote external device 3286 may be accessible to an appropriately authorised person such as a clinician.

The local external device 3288 may be a personal computer, mobile phone, tablet or remote control.

### 4.4.2.9 Output Devices Including Optional Display, Alarms

An output device 3290 in accordance with the present technology may take the form of one or more of a visual, audio and haptic unit. A visual display may be a Liquid Crystal Display (LCD) or Light Emitting Diode (LED) display.

### 4.4.2.9.1 Display Driver

A display driver 3292 receives as an input the characters, symbols, or images intended for display on the display 3294, and converts them to commands that cause the display 3294 to display those characters, symbols, or images.

### 4.4.2.9.2 Display

A display 3294 is configured to visually display characters, symbols, or images in response to commands received from the display driver 3292. For example, the display 3294 may be an eight-segment display, in which case the display driver 3292 converts each character or symbol, such as the figure “0”, to eight logical signals indicating whether the eight respective segments are to be activated to display a particular character or symbol.

### 4.5 Air Circuit

An air circuit 3170 in accordance with an aspect of the present technology is a conduit or a tube constructed and arranged to allow, in use, a flow of air to travel between two components such as RPT device 3000 and the patient interface 2000 or 2800.

In particular, the air circuit 3170 may be in fluid connection with the outlet of the pneumatic block 3020 and the patient interface. The air circuit may be referred to as an air delivery tube. In some cases there may be separate limbs of the circuit for inhalation and exhalation. In other cases a single limb is used.

In some forms, the air circuit 3170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 3170. The heating element may be in communication with a controller such as a central controller 3230. One example of an air circuit 3170 comprising a heated wire circuit is described in U.S. Pat. No. 8,733,349, which is incorporated herewithin in its entirety by reference.

### 4.6 Humidifier

### 4.6.1 Humidifier Overview

In one form of the present technology there is provided a humidifier 4000 (e.g. as shown in FIG. 5A) to change the absolute humidity of air or gas for delivery to a patient relative to ambient air. Typically, the humidifier 4000 is used to increase the absolute humidity and increase the temperature of the flow of air (relative to ambient air) before delivery to the patient's airways.

The humidifier 4000 may comprise a humidifier reservoir 4110, a humidifier inlet 4002 to receive a flow of air, and a humidifier outlet 4004 to deliver a humidified flow of air. In some forms, as shown in FIG. 5A and FIG. 5B, an inlet and an outlet of the humidifier reservoir 4110 may be the humidifier inlet 4002 and the humidifier outlet 4004 respectively. The humidifier 4000 may further comprise a humidifier base 4006, which may be adapted to receive the humidifier reservoir 4110 and comprise a heating element 4240.

### 4.6.2 Humidifier Components

### 4.6.2.1 Water Reservoir

According to one arrangement, the humidifier 4000 may comprise a water reservoir 4110 configured to hold, or retain, a volume of liquid (e.g. water) to be evaporated for humidification of the flow of air. The water reservoir 4110 may be configured to hold a predetermined maximum volume of water in order to provide adequate humidification for at least the duration of a respiratory therapy session, such as one evening of sleep. Typically, the reservoir 4110 is configured to hold several hundred millilitres of water, e.g. 300 millilitres (ml), 325 ml, 350 ml or 400 ml. In other forms, the humidifier 4000 may be configured to receive a supply of water from an external water source such as a building's water supply system.

According to one aspect, the water reservoir 4110 is configured to add humidity to a flow of air from the RPT device 3000 as the flow of air travels therethrough. In one form, the water reservoir 4110 may be configured to encourage the flow of air to travel in a tortuous path through the reservoir 4110 while in contact with the volume of water therein.

According to one form, the reservoir 4110 may be removable from the humidifier 4000, for example in a lateral direction as shown in FIG. 5A and FIG. 5B.

The reservoir 4110 may also be configured to discourage egress of liquid therefrom, such as when the reservoir 4110 is displaced and/or rotated from its normal, working orientation, such as through any apertures and/or in between its sub-components. As the flow of air to be humidified by the humidifier 4000 is typically pressurised, the reservoir 4110 may also be configured to prevent losses in pneumatic pressure through leak and/or flow impedance.

### 4.6.2.2 Conductive Portion

According to one arrangement, the reservoir 4110 comprises a conductive portion 4120 configured to allow efficient transfer of heat from the heating element 4240 to the volume of liquid in the reservoir 4110. In one form, the conductive portion 4120 may be arranged as a plate, although other shapes may also be suitable. All or a part of the conductive portion 4120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 4.6.2.3 Humidifier Reservoir Dock

In one form, the humidifier 4000 may comprise a humidifier reservoir dock 4130 (as shown in FIG. 5B) configured to receive the humidifier reservoir 4110. In some arrangements, the humidifier reservoir dock 4130 may comprise a locking feature such as a locking lever 4135 configured to retain the reservoir 4110 in the humidifier reservoir dock 4130.

### 4.6.2.4 Water Level Indicator

The humidifier reservoir 4110 may comprise a water level indicator 4150 as shown in FIG. 5A-5B. In some forms, the water level indicator 4150 may provide one or more indications to a user such as the patient 1000 or a care giver regarding a quantity of the volume of water in the humidifier reservoir 4110. The one or more indications provided by the water level indicator 4150 may include an indication of a maximum, predetermined volume of water, any portions thereof, such as 25%, 50% or 75% or volumes such as 200 ml, 300 ml or 400 ml.

### 4.7 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Tᵢ 1.6 s, peak inspiratory flow rate Qₚₑₐₖ 0.4 L/s, exhalation time Tₑ 2.4 s, peak expiratory flow rate Qₚₑₐₖ−0.5 L/s. The total duration of the breath, Tₜₒₜ, is about 4 s. The person typically breathes at a rate of about 15 breaths per minute (BPM), with Ventilation Vent about 7.5 L/min. A typical duty cycle, the ratio of Tᵢ to Tₜₒₜ, is about 40%.

### 4.8 Patient Tracker

In some forms of the present disclosure, a system 5000 may be provided for measuring parameters, i.e., characteristics of a patient when they are off therapy such as while they are awake, or before and after therapy sessions. The system may be configured to provide a patient 5001 with ongoing monitoring of their waking physiological parameters, i.e., an ‘awake state’ to determine, i.e., quantify, whether their sleep quality improves as a result of respiratory therapy, e.g., PAP therapy. In this regard, the system 5000 may be considered a patient tracker 5000 configured to sense a patient's physiological state. The patient tracker 5000 can acquire sensor data off therapy, e.g. during the day if the patient is under PAP therapy, rather than at night, and may also be further configured to acquire sensor data whilst the patient is on therapy if required.

Referring to FIGS. 7 and 8, the system 5000 may be implemented in the form of a wearable device, such as the ‘earbud’ type wearable device shown wearable with respect to a person's ear canal, ear lobe or behind the person's ear. In this regard, the patient tracker 5000 may be considered a patient tracking device 5000 (also referred to as device 5000).

The patient tracking device 5000 may be configured to measure daytime activities and physiological characteristics, i.e., conditions, of the patient. For example, the system may be configured to measure activities such as: a distance travelled by the patient; a number of steps/paces walked, run, climbed etc.; a type, duration, intensity, etc., of physical activity; time spent standing. Alternatively, the patient tracking device may receive data pertaining to daytime activities from another device, such as another wearable device.

The daytime activities set forth above may influence the physiological characteristics of the patient. For example, a patient travelling a distance may have an elevated heart rate, increased breath rate, etc. The physiological characteristics that can be measured by the system include: a respiration rate, variability of respiration, etc.; a heart rate, variability of heart rate, etc.; a magnitude of calories burned; blood oxygen saturation; electrodermal activity (i.e., skin conductance or galvanic skin response); or any combination thereof.

Referring firstly to FIG. 7, the device 5000 may be used by itself, or independently of, i.e., without a respiratory pressure therapy (RPT) device. In particular, the device is shown without a patient interface 5008. In this form, the patient may, for example, wear the device by itself during daytime activities such as walking, running, etc.

By comparison, and as shown in FIG. 8, the device 5000 may also be used together with an RPT device. In the form shown, the device is worn together with a patient interface 5008 (as part of the RPT). The patient may wear the device in this way, i.e., with the patient interface 5008, while they sleep for recording data while also receiving respiratory therapy.

The patient tracker 5000 may include a control system and a memory device, and one or more sensors. As set forth previously, the system 5000 may be integrated into a wearable device such as an earpiece, i.e., an ‘earbud’, or in other forms not shown, integrated into a watch, a ring, an earring, a bracelet, a necklace, an item of clothing, etc., as set forth in more detail later.

As shown in FIGS. 7 and 8, the device 5000 comprises a body 5002 for housing the control system, memory device, sensors, batteries (rechargeable or replaceable), etc. An ear hook 5004 is provided for locating, i.e., attaching, mounting, etc., the device 5000 about the patient's ear. In particular, the ear hook is configured, i.e., shaped, to locate and removably secure behind the patient's external ear (i.e., auricle/pinna). The ear hook may be made from a flexible, soft material such as silicone rubber or polyester so as to prevent contacted pressure sores if the patient is wearing the device 5000 while sleeping, i.e., side-sleeping.

The body 5002 is configured to locate within the patient's ear for transmitting audio (i.e., sound) into the ear for the patient to hear. At least a portion of the body 5002 may be configured to releasably secure within at least a portion of the patient's external auditory canal. In this regard, the body of the device may be shaped similar to a traditional earbud used for transmitting audio into a patient's ear.

The control system may include one or more processors. The control system generally controls the various components of the system and analyses data obtained or generated by components such as the sensors or other wearable devices.

The patient tracker 5000 may be configured to receive the physiological data about the patient from the one or more sensors. In some forms, the patient tracker may also be configured to receive environmental data from the one or more sensors. The environmental data may relate to environmental conditions surrounding the patient (i.e., the environmental data being related to the patient), such as temperature, humidity, etc. In either form of data, i.e., physiological or environmental, the data may be stored in the memory device and analysed by the processor(s) of the control system.

Advantageously, measuring and recording data relating to the environmental conditions surrounding the patient may allow the device 5000 to accommodate for environmental conditions that influence the physiological conditions of the patient. For example, if the humidity and temperature of air surrounding the patient is high, the patient may fatigue more rapidly when e.g., walking, than in colder, less humid conditions. In effect, environmental conditions (such as high humidity and temperature) may inadvertently indicate the patient is fatigued as a result of e.g., a lack of sleep. Hence, allowing the device 5000 to accommodate for such environmental conditions means that indications of the patient's sleep quality can be more accurately presented to the patient.

The one or more sensors may include, for example, a pressure sensor, a flow rate sensor, temperature sensor, a motion sensor, a microphone, a speaker, a radiofrequency (RF) receiver, a RF transmitter, a camera, an infrared (IR) sensor, a photoplethysmogram (PPG) sensor, an electrocardiogram (ECG) sensor, an electroencephalography (EEG) sensor (using a dry-electrode), a capacitive sensor, an electromyography (EMG) sensor, an oxygen sensor, an analyte sensor, a moisture sensor, a light detection and ranging (LiDAR) sensor, an electrooculography (EOG) sensor, a peripheral oxygen saturation (SpO₂) sensor, a galvanic skin response (GSR) sensor, a carbon dioxide (CO₂) sensor, or any combination thereof.

For example, an optical sensor using red, infrared, and/or green, could be used to calculate a photoplethsmogram. Subsequently, parameters such as pulse rate (PR), pulse rate variability (PRV), SpO2 can be determined. If respective sensors are placed on a periphery of the user, e.g., at their skin, the peripheral arterial tone may also be measured.

Generally, the types of sensors (set forth above) utilised in the patient tracker 5000 may vary according to the physiological and/or environmental data being generated. For example, when the patient tracker 5000 is integrated into an item of clothing, it may comprise the electromyography (EMG) sensor for detecting electrical signals generated by muscles. Alternatively, the EMG sensor may not be utilised when the patient tracker is integrated into a ring. In any case, each of the one or more sensors may be configured to output sensor data that is received and stored in the memory device of the patient tracker 5000.

In some forms of the device 5000, one or more of the sensors set forth above may be configured to contact the patient's skin. In this regard, the sensors may be located on an externally facing surface of the body 5002 or the ear hook 5004, so as to be in contact with the patient's skin when in use. This allows e.g., the galvanic skin response (GSR) sensor to measure changes in sweat gland activity on the skin. In another example, the one or more sensors, e.g., optical sensor, may be located on the ear hook so as to contact an area of skin between the patient's auricle/pinna and hairline.

Other forms of the sensors may be configured for mounting internally to the body 5002 and ear hook 5004, such as the motion sensor. In this case, for example, the motion sensor may be integrated within the body of the device and configured to measure a patient's head movement.

As set forth above, the one or more sensors of the patient tracker 5000 can be configured to determine an awake state of the patient. The patient tracker may utilise the physiological, activity and environmental data generated from the sensors to determine how ‘awake’, i.e., alert, the patient is for a duration of non-sleep, e.g., during the daytime. For example, the device 5000 may be configured to measure a patient's heart rate and EEG during the daytime. Based on the physiological data generated from variations in the heart rate and EEG measurements, the system 5000 may indicate how awake the patient is, e.g., if the patient is lethargic and has an unfocussed attention during the daytime.

In order to determine an awake state, including stages of a sleep (such as NREM (N1, N2, N3/SWS) or REM), data may be feed into an artificial Intelligence (AI) or Machine Learning (ML) model. This model may be trained on the IMU and PPG signals, or pre-processed parameters of those.

Breathing/respiration signal related parameters can include: variability of breathing rate throughout the day and/or night (the variability being characteristic of the person)—this can be inter-breath or over longer timescales—e.g., 30, 60, 90 sec or much longer periods; the stability over time (related to the variability); the standard deviation of breathing rate; the depth of respiration (shallow, deep etc.), and relative amplitude of adjacent breaths; the mean or average value of the breathing rate; the trimmed mean (e.g., at 10%) to reject outliers; wake or Asleep (i.e., the detected sleep state of the person); surges (sudden accelerations or decelerations) in breathing rate seen during quiet periods and during REM sleep; median (50th percentile); interquartile range (25th-75th percentile); 5th-95th percentile; 10th-90th percentile; shape of histogram; skewness; kurtosis; peak frequency over time; ratio of second and third harmonics of peak frequency; percentage of valid data (Valid Physiologically Plausible Data); autocorrelation of the individual signals; characteristic patterns in the spectrogram; wake or asleep; relative percentage of REM and deep sleep.

Cardiac/heart signals can be processed to produce features such as: heart rate variability HRV (inter beat (e.g., as derived from the Ballistocardiogram) and over longer defined moving windows—e.g., 30, 60, 90 sec); variability over time (interbeat/breath variability)); mean; trimmed mean (10%); standard deviation; median (50th percentile); interquartile range (25th-75th percentile); 5th-95th percentile; 10th-90th percentile; shape of histogram; skewness; kurtosis; stability over time; peak frequency over time; ratio of second and third harmonics of peak frequency; percentage of valid data (Valid Physiologically Plausible Data), wake or asleep; autocorrelation of the individual signals; characteristic patterns in the spectrogram.

Cardiorespiratory signals can be formed, such as: magnitude square cross spectral density (in a moving window); cross coherence; respiratory sinus arrhythmia peak; low frequency (LF)/high frequency (HF) ratio to indicate autonomic nervous system parasympathetic/sympathetic balance (LF is often defined as around 0.04-0.15 Hz, whereas HF is around 0.15-0.4 Hz); the cross correlation; cross coherence (or cross spectral density) of the heart and breathing signal estimates; non-linear estimates such as entropy measures; the characteristic movement patterns over longer time scales, i.e., the statistical behaviour observed in the signals; patterns of movement during detection of and comparison of these heart and breathing signals (e.g., during sleep, some people may have more restful and some more restless sleep).

Based on the determination of a patient's awake state, the patient tracker 5000 may provide the patient with an indication of how effective their respiratory therapy, e.g., PAP therapy, is at improving their sleep quality. For example, in the case where physiological data indicates the patient is lethargic and unfocussed, such an indication may be correlated with a low efficacy of the patient's PAP therapy. Conversely, in the case where physiological data indicates the patient has improved capacity for daytime activities, e.g., a lower resting heart rate, etc., such an indication may be correlated with a high efficacy of the patient's PAP therapy. As set forth in more detail later, the patient tracker may be configured to alert the patient of such indications, e.g., notifications that e.g., a morning run, positively impacted their sleep.

The patient tracker 5000 may be configured to measure an efficacy of respiratory therapy by recording a baseline measure of ‘off therapy’ physiological data and comparing this to an ‘on therapy’ measure of physiological data. According to the changes detected in the measured data, the patient tracker may advise the patient of either improvements to their sleep performance, or deteriorations to their sleep performance.

In a variation, the patient may be advised of improvements that occur in their ability to undertake daytime activities, such as capacity for exercise, that are a result of their corresponding improvements to their sleep performance. Conversely, the patient tracker can be configured to notify the patient of a deteriorated capacity to perform daytime activities as a result of a corresponding deterioration in their sleep performance. Advantageously, notifying a patient of said changes to either their sleep performance or capacity for daytime activities can allow a patient to understand an impact of their respiratory therapy.

As part of providing the patient with an indication of how effective their respiratory therapy is, the patient tracker 5000 may also be configured to record, i.e., ‘timestamp’ events associated with the patient's sleep periods. For example, the various sensors of the patient tracker 5000 may be configured to record a time that the patient wakes after a period of sleep, times when the patient wakes during a period of sleep (i.e., a rate of sleep disturbances), a time that the patient exits the bed, a time that the patient enter the bed, etc. These events may be utilised, i.e., analysed, together with other sensor data gathered about the patient, to determine how ‘awake’ the patient may be as a result of their e.g., PAP therapy.

Advantageously, data relating to e.g., when a patient wakes, may be longitudinally recorded so as to determine sleeping patterns of the patient. This information may be processed and utilised to inform the patient of e.g., whether they are ready for sleep; whether they are sleeping well; whether they should expect to feel tired during their waking hours, etc. Ultimately, the patient tracker 5000 may provide the patient with an indication of how effective their respiratory therapy has been.

Set forth below are some further examples of sensors that may be used with the patient device 5000, and their application for use with the patient device 5000.

In some forms of the patient tracker 5000 where the motion sensor is utilised (as set forth previously), the motion sensor may generate data relating to specific movements of the patient, such as exercise (e.g., running), or other body (e.g., limb) movements. These movements may be utilised to determine the patient's awake state. For example, a patient's limb movements may be analysed and determined as being slow relative to a standard measurement of the patient's ‘normal’ movements.

While the motion sensor is described above in broad terms, the motion sensor may be specifically one or more inertial sensors, such as accelerometers, gyroscopes, and magnetometers. These types of motion sensors may be selected, i.e., utilised according to their optimal use-case.

In some forms of the motion sensors, the motion sensors may be configured to detect motion or acceleration associated with arterial pulses, such as pulses in or around the face of the patient and in particular, those proximal to the patient tracking device 5000, e.g., the body 5002. The motion sensors in this form may be configured to detect features of the pulse shape, speed, amplitude, or volume that may be analysed to indicate qualities of a patient's awake state.

In other forms, an EEG sensor may also be provided in the patient tracker 5000 for measuring physiological data relating to the patient's brain. The EEG sensor may include one or more dry electrodes positioned on or around the scalp of the patient. In this form, the EEG may locate within, or extend from, a portion of the ear hook 5004 or body 5002. For this reason, the EEG sensor is optimally utilised when the patient tracker is implemented as an earpiece, as shown in FIGS. 7 and 8, such that the external surfaces of the body 5002 and ear hook 5004 may be in contact with the patient's scalp.

Depending on the placement of the EEG sensors, it may be possible to detect EEG slowing during the daytime (such as a higher ratio of delta and theta frequencies to alpha and beta frequencies) and relate this daytime slowing to greater daytime sleepiness. Thus, it may be possible to avoid asking a patient if they have ‘daytime sleepiness’, but rather, derive it from EEG slowing vs. a baseline and relate this to a reduced movement of the patient (detected from e.g., an accelerometer).

In forms where a PPG sensor is provided to measure, e.g., a heart rate, the patient tracker is optimally configured to contact the patient's skin. In this form, the patient tracker may be integrated into a piece of clothing to optimally generate data relating to e.g., a heart rate pattern, a heart rate variability, a cardiac cycle, respiration rate, estimated blood pressure, or any combination thereof.

When the patient tracker is integrated into an earbud/earpiece as shown in FIGS. 7 and 8, a speaker 5006 may be provided for outputting (i.e., generating) audio. The audio, i.e., generated sounds, are configured to be projected into the patient's ear so as to be heard by the patient. For example, the patient tracker 5000 may be configured as a type of ‘earphone’ to play music for a patient to listen to during the day. In another example, the patient tracker 5000 may be configured to sound an alarm for waking the patient from sleep, reminding them of an event (e.g., a calendar event). In yet a further example, the device 5000 may assist in relaxation of the patient prior to sleep by playing controlled breathing audio cues. In yet a further example again, the device 5000 may also provide hearing assistance, whereby the device may be coupled with a smartphone to generate audio, amplify audio, etc.

In some implementations, the speaker 5006 may be used together with, or substituted by, a bone conduction speaker. In this form, the bone conduction speaker is not configured to generate audio for the patient to hear via their ears, rather, the speaker generates vibrations that are configured to penetrate the patient's temporal bones. In variations, an audio and bone conduction speaker may be configured for use together.

In some further implementations, the speaker 5006 may be a noise cancelling speaker for assisting in reduction of background noises. Advantageously, this may be used prior to sleep, for reducing background noises that may otherwise hinder sleep.

In either form of the speaker, e.g., as a speaker, bone conduction speaker, noise cancelling speaker, etc., the patient tracking device 5000 may be coupled (i.e., wired or wirelessly) to a computing device, e.g., a mobile phone, for playing music or otherwise generating the sounds for the patient to hear. In the case of a wireless connection, the patient tracking device 5000 may be configured to communicate through various communication protocols, such as, WiFi, Bluetooth, etc. The patient tracking device may thereby include an antenna, a receiver, a transmitter, a transceiver, or any combination thereof for communicating with wirelessly with a computing device.

The computing device may be configured to operate, i.e., ‘run’ software configured to communicate with the patient tracking device 5000. In forms where the computing device is a mobile phone or tablet, the software may be configured as a mobile application, i.e., ‘app’, allowing the patient to control operation of the patient tracking device via the mobile device.

The computing device may be used as a way to display information about the patient's awake state. In other forms, the computing device may also be configured to process (via one or more processors) data generated from the patient tracking device 5000. In further forms, the computing device may be configured to receive input from the patient for controlling operation of the patient tracking device. As set forth above, the input of the patient may relate to the patient configuring the patient tracking device to send diary alarms, or in other cases, to select music to listen to (via the speakers).

In some forms of the patient tracking device, the patient may input information into the computing device, i.e., via the software, for determining, at least in part, the awake state of the patient. That is, the patient may ‘self-report’ information that may not be sensed, per se, but be provided by the patient to be considered together with physiological and/or environmental data generated by the sensors. The combination of self-reported data and sensed data may be analysed to determine a patient's awake state.

The self-reported information input by the patient may include demographic information, biometric information, medical information such as medications, etc., diet(s), subjective stress level of the patient, subjective fatigue level of the patient, subjective health status of the patient, a recent life event experienced by the patient, or any combination thereof.

In the case of demographic information, the patient may provide their age, gender, race, employment status, socioeconomic status, etc. In the case of the medical information, the patient may provide information relating to one or more medical conditions, medication usage, etc.

Referring now to FIG. 8, the patient tracking device 5000 may be configured for use with respiratory therapy, e.g., a respiratory pressure therapy (RPT) device (as set forth previously). The RPT device may include the patient interface 5008, a conduit 5010, a mask 5012 and a positioning and stabilising structure 5014. It is noted that, although a nasal prongs mask is shown in FIG. 8, other types of masks may be utilised, such as a full-face mask, nasal mask, oro-nasal mask, etc.

As set forth above, the patient tracker 5000 may provide the patient with ongoing monitoring of their ‘awake’ state and provide feedback to the patient regarding any differences detected between ‘on’ and ‘off’ therapy. In other words, the patient tracker 5000 may indicate changes in the patient's sleep performance after they begin respiratory therapy and, in effect, indicate to the patient how effective their use of respiratory therapy has been.

The patient tracker 5000 may be configured to correlate changes in a patient's daytime activities with their adherence/compliance to e.g., CPAP therapy. For example, in patients having symptoms such as chronic fatigue, daytime sleepiness, cognitive impairment, etc., the patient tracker (as set forth previously) may be configured to monitor for improvements in such symptoms. The patient tracker may be configured to determine correlations between these improvements, i.e., changes, and the patient's adherence/compliance to CPAP therapy. These correlations may be reported, i.e., communicated as feedback to the patient, so that the patient is aware of the positive impact their adherence/compliance to CPAP therapy has on their capacity for performing daytime activities.

The patient tracker 5000 may be configured to interrelate a specific respiratory therapy, e.g., CPAP and a deterioration of healthy behaviours or an improvement of healthy behaviours. That is, the patient tracker 5000 may also be configured to monitor and report to a patient their unhealthy, i.e., ‘bad’ behaviours which may occur as a result of sleep related breathing disorders.

For example, patients having sleep apnea for an extended period of time prior to diagnosis may develop unhealthy behaviours, such as lack of exercise, bad sleep habits, etc., which may persist even after commencing respiratory therapies, e.g., CPAP. The sensor(s) and self-reported information input into the patient tracker 5000 may be used to monitor and report to the patient such behaviours. Reporting these behaviours as ‘feedback’ to the patient may assist the patient to change, i.e., ‘re-train’such behaviours. Advantageously, re-training the patient to remove said unhealthy behaviours can positively impact their respiratory therapy, in addition to reducing the patient's risk of comorbidities.

In this regard, the patient tracker 5000 can provide the patient with a ‘complete treatment’ for their sleep related breathing disorder(s). That is, in addition to opening the patient's airways via, e.g., PAP therapy, the patient tracker can identify and treat unhealthy behaviours that are symptomatic of the sleep related breathing disorder. Advantageously, this can motivate a patient to be more adherent/compliant to respiratory therapy.

In some forms, the patient tracker 5000 may be configured to provide the patient with detailed correlations of their improved daytime activities and corresponding compliance to respiratory therapy. For example, the patient tracker 5000 may be configured to correlate a patient's use of CPAP therapy during a sleep period, with the patient being able to run a larger distance the following day, or the patient having a lower resting heart rate, etc.

The patient tracker 5000, as set forth above, can be configured to improve a patients adherence/compliance by behavioural intervention. That is, the patient tracker 5000 can be configured to allow a patient to break, i.e., intervene, particular habits that are associated with their sleep related breathing disorder(s).

In some forms, a patient's compliance/adherence to a respiratory therapy may also be detected by measurements taken by the one or more sensors of the patient tracker 5000. For example, the patient tracker may include an EEG configured to measure daytime markers of a patient's increased alertness. Such markers may be compared against ‘normal’ measures of the patients' alertness, such that an indication of the patients' improved alertness can be determined. This can indicate an improved efficacy of the respiratory therapy, and in turn, indicate the patients' adherence/compliance to therapy. Advantageously, use of the sensors to automatically detect efficacy and therapy adherence/compliance means that the patient may not be required to monitor their perceived ‘daytime sleepiness’, i.e., lethargy or reduced alertness to determine an efficacy of their respiratory therapy.

Furthermore, utilising the EEG for compliance indications may also allow for a detection of impaired cognitive function. That is, detection of a patient's alertness may be used as a proxy for an assessment of their cognitive function.

In some forms, the patient tracking device 5000 may be coupled with the respiratory pressure therapy (RPT) device to monitor the patient's sleep state during periods of sleep. In this form, the sensors of the patient tracking device 5000 may be used together with the sensors of the RPT device (i.e., optionally located in the patient interface 5008, flow generator, or other component of the RPT device), for detecting e.g., states of a sleep cycle. In some forms, the data collected may be used to inform the patient of how effective their respiratory therapy has been, and in other forms the data may be additionally or alternatively used to adjust the delivery of respiratory therapy, e.g., pressure, flow rate, etc.

In some forms, a microphone may also be provided to the patient tracking device 5000 to measure a patient's breathing during sleep. In this form, the microphone may be located proximal to the patient's mouth and/or nose, and so accurately record breathing sounds. A detection of abnormal breathing may be indicative of a sleep apnoea, whereby the patient tracking device may be used together with the RPT to adjust therapy, e.g., pressure, flow, etc., for stimulating a change in the patient's breathing.

In some further forms, the motion sensor described previously may be utilised during a patient's sleeping period to detect movements of the patient. For example, a number of movements during a sleep period may be detected, and used to provide an indication of e.g., a disturbed sleep. In some forms, the data collected from the motion sensor may be fused, i.e., coupled, combined, integrated, etc., with flow data collected from the RPT device. This combination of data may be used to improve sleep/wake classification, i.e., determination of a patient's awake state.

In other forms, the patient tracker 5000 may be configured to monitor the patient's sleep state without being coupled to the RPT device. In this form, the patient tracker 5000 may be configured to detect and record physiological and/or environmental data as it would when coupled with the RPT device. However, rather than adjust operation of the RPT device, the patient tracker 5000 in this form would utilise the data recorded to inform the patient of their sleep performance, e.g., apnoea events, etc.

In some further forms where the patient tracking device 5000 is used without being coupled to the RPT device, the data collected during a sleeping period may be implemented as a change to respiratory therapy at a later date. That is, the data collected when the patient is not wearing the patient interface 5008 may be used to adjust therapy the next time the patient wears the patient interface 5008.

In some further forms again, the patient tracking device 5000 may be configured to intermittently couple with the RPT device so as to communicate with the RPT device. The patient tracking device in this form may be configured to operate both together with the RPT device, and independently of the RPT device. That is, when the patient is near the RPT device, the patient tracker 5000 may be able to connect (e.g., wirelessly) with the RPT device. When the patient is away from the RPT device, e.g., walking outside, the patient tracker may be able to operate independently of the RPT device.

For example, the patient tracker 5000 operating independently may be able to temporarily record and store data from the sensors for later communicating said data to the RPT device when the patient tracker 5000 is proximal to the RPT device.

In this form, the patient tracking device 5000 may be worn together with the patient interface 5008 in some instances, e.g., during sleep, and in other instances the patient tracking device may not be worn with the patient interface 5008, e.g., when a patient leaves their home. In some cases, missing data i.e., data which is not collected by either the RPT device or patient tracking device 5000, may be collected from an alternative data source, such as a wrist worn accelerometer or HR sensor. For example, the device 5000 may be coupled with an external device such as a smart watch, or a ‘smart hub’ for collecting data that may not be captured by the device 5000 or the RPT device.

In some forms, the computing device, e.g., mobile device, as set forth previously may be configured to connect with the patient tracker 5000 when the patient tracker in not coupled with the RPT device. In this regard, the device may be configured to log and process data within its memory, without requiring a wireless connection for a period of time.

In some forms, the patient tracker 5000 may be utilised for detecting and diagnosing a patient with an un-treated sleep related breathing disorder. The patient tracker 5000 used in this form may allow a patient to determine whether they require respiratory therapy e.g., PAP therapy, positional therapy, insomnia treatment, etc. In this form, the sensors (as set forth previously) may be configured to register (i.e., detect) a sleep event that is indicative of a sleep related breathing disorder.

In forms whereby the patient tracker 5000 is configured for detecting and diagnosing a patient with a sleep related breathing disorder, the patient tracker may be utilised to monitor a patient's daytime activities to determine indications of sleep related breathing disorders. For example, a patient may develop unhealthy behaviours, such as lack of exercise, bad sleep habits, etc., that may be detected and utilised as an indicator of insomnia, etc.

In a variation, the patient tracker 5000 may be utilised for detecting a patient with an under-treated sleep related breathing disorder. That is, a patient having already been diagnosed with a sleep related breathing disorder, but is not receiving effective therapy. In this case, the patient tracker may be configured to monitor e.g., heart rate variability for indicating whether the patient is under-treated. In response, the patient tracker may be configured to provide the patient with an indication of how to adjust therapy during the night, or alternatively, the patient tracker may be configured to automatically adjust a respiratory therapy device (as set forth previously) to appropriately treat the under-treated disorder.

In some forms, the patient tracker 5000 can also be configured for detecting and monitoring for comorbidities of sleep apnoea. For example, the sensor(s) set forth above may be configured for detecting diabetes, heart failure, stroke and obesity.

The patient tracker 5000 may be used in conjunction with a respiratory therapy system, to aid in adjusting or tuning the respiratory therapy which is delivered to the patient. The patient tracker 5000 may include sensors which are configured to acquire data in relation to the patient when a body of the patient tracker 5000 is located in the patient's ear. The acquired data may include physiological data including data from physiological sensors and electrodes mentioned above, activity data which in some forms may comprise position data of the patient. The acquired data may further include an environmental data as mentioned previously.

With reference to FIG. 9, in one variation, a respiratory therapy system 6000 includes a processing arrangement 6002 which may comprise one or more processors, memory 6004 having stored therein a therapy control program for execution by the one or more processors. The therapy control program is configured to control operation of the respiratory therapy system 6000 to deliver therapy via a respiratory therapy delivery arrangement 6008. The control may reside in the adjustment or tuning of the respiratory therapy. This may include adjusting one or more parameters of the therapy, or adjusting the duration or timing of the application of the respiratory therapy. The respiratory therapy system 6000 is adapted for data communication with the wearable device and in this embodiment includes a communication module 6006. The respiratory therapy system 6000 is configured to receive data from the patient tracker 5000 when the data communication is established with the wearable device. The one or more processors 6002, the memory device 2004, and the communication module 6006, may be collocated with the therapy delivery arrangement 6008, or it may be located in a housing separate to the therapy delivery arrangement 6008 as shown in FIG. 9.

An example of the operation which execution of the therapy control program implements is shown in FIG. 10. At step 7002 a data communication session between the processor 6002 and the patient tracker 5000 is established. At step 7004 data is received at the therapy system 6000. The data which is received at the respiratory therapy system 6000 may comprise the sensor data acquired by the patient tracker 5000, or an analysis result from analysing the acquired sensor data, or both. At step 7006, the therapy control program determines an adjustment to the respiratory therapy on the basis of the received data. In some embodiments, at step 7008, the processor may store the received data in the memory for a predetermined period of time, prior to determining the adjustment, so that the determination may make use of longitudinal data acquired over the period of time. At step 7010, the determined adjustment is stored in the memory, and at step 7012 applied to the respiratory therapy during the next therapy session.

The therapy control program may be configured to determine a correlation between the received data and a data in relation to the respiratory therapy, and then determine the adjustment on the basis of the correlation. For example, the therapy control program may determine whether there is any statistically significant correlation between the acquired data and any aspect of the respiratory therapy. Identification of such a correlation may be used in future to set a model for adjusting the therapy on the basis of the received data.

The determination of the adjustment may be at least partially based on a comparison between received data corresponding to sensor data acquired over a first time period with received data corresponding to sensor data acquired over a second time period. The first time period may be during an off-therapy period when the patient is not undergoing any therapy, such as before the patient is put on the respiratory therapy or when the patient is not adhering to the respiratory therapy. The second time period may be during an on-therapy period where the patient is adhering to respiratory therapy. Adherence may be defined as having a minimum duration of the therapy being turned on nightly for at least a number of days, or more generally, at or above a frequency as calculated over a predetermined number of days. The level of adherence may be represented by a compliance rate. In another variation, the two time periods may both be “on-therapy” periods, with the patient's therapy compliance rate with a required range. This can be useful in assessing whether the therapy is leading to improvements in physiological or activity data, or inferred information such as the patient's mental acuity. In another variation the two time periods may both be on-therapy periods. Changes observed in the analysis data may be used to compare with compliance metrics from the two periods, to determine a correlation between compliance and the physiological or activity parameters for the patient.

In a variation, the time periods may be shorter periods rather than periods extending a number of days. For example, the first period of time may be a day time or awake period when the patient is awake, and the second period of time may be a night time or sleep period after the first period of time. In this case, the analysis data from the sensor data acquired during the awake period may be used to determine how to set or tune the therapy setting during the second period of time. In this case the patient may not necessarily be wearing the wearable device during the therapy session. However the patient may alternatively be wearing the wearable device during the second period of time also, in which case further in-therapy adjustments may be performed. Alternatively or additionally, the performance or efficacy of the therapy which now has its setting tuned in accordance with the determination made from the data acquired during the first period of time, may be determined from the sensor data acquired during the second period of time. The performance or efficacy data may be used to evaluate the effectiveness of the tuning or adjustment made to the respiratory therapy.

With reference to FIG. 10, in a variation, a method of determining an adjustment to a respiratory pressure therapy for a patient includes, at step 7100, acquiring sensor data using one or more sensors in a patient tracker in the form of a wearable device adapted to be located in an ear of a patient, when the wearable device is so located. At step 7102, the acquired data is stored in a memory device. This memory device may be the memory device local to the wearable device, or may be a memory device within the respiratory therapy system. At step 7104, the stored data is processed to determine analysis data, comprising one or more physiological or activity parameters. At step 7106, one or more adjustments to the therapy is determined on the basis of the analysis data.

Steps 7100 and 7102 may be repeated over at least a first period of time and a second period of time. Having the data from the two time periods allows for a longitudinal comparison between sensor data acquired during the two periods, or the analysis data obtained based on the sensor data acquired during the two periods. In variations where the wearable device also acquires environmental data, the determination of the analysis data may involve modulating the sensor data, or the calculated physiological or activity parameters, based on one or more environmental data.

In an embodiment, step 7100 is performed to acquire sensor data over an awake period when the patient is not asleep. This is then stored, and then processed in order to determine a respiratory therapy setting, or to make a change or adjustment to tune an existing setting to be applied during an upcoming therapy session, e.g., during the patient's sleep after the day time during which the sensor data was acquired.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

4.11 REFERENCE SIGNS LIST

| | |
|---|---|
| Patient | 1000 |
| bed partner | 1100 |
| patient interface | 3000 |
| seal - forming structure | 2100 |
| cushion module | 2150 |
| plenum chamber | 2200 |
| Chord | 2210 |
| superior point | 2220 |
| inferior point | 2230 |
| plenum chamber inlet port | 2254 |
| positioning and stabilising structure | 2300 |
| Headgear | 2302 |
| four-point headgear | 2302-1 |
| two-point headgear | 2302-2 |
| Strap | 2310 |
| Inlet | 2332 |
| Tube | 2350 |
| Vent | 2400 |
| Groove | 2416 |
| connection port | 2600 |
| Elbow | 2610 |
| forehead support | 2700 |
| RPT device | 3000 |
| external housing | 3010 |
| upper portion | 3012 |
| Portion | 3014 |
| Panel | 3015 |
| Chassis | 3016 |
| Handle | 3018 |
| pneumatic block | 3020 |
| air filter | 3110 |
| inlet air filter | 3112 |
| outlet air filter | 3114 |
| Muffler | 3120 |
| inlet muffler | 3122 |
| outlet muffler | 3124 |
| pressure generator | 3140 |
| Blower | 3142 |
| Motor | 3144 |
| anti - spill back valve | 3160 |
| air circuit | 3170 |
| air circuit | 3171 |
| supplementary gas | 3180 |
| electrical components | 3200 |
| single Printed Circuit Board Assembly PCBA | 3202 |
| power supply | 3210 |
| input device | 3220 |
| central controller | 3230 |
| Clock | 3232 |
| therapy device controller | 3240 |
| protection circuits | 3250 |
| Memory | 3260 |
| Transducer | 3270 |
| pressure sensor | 3272 |
| flow rate sensor | 3274 |
| motor speed transducer | 3276 |
| data communication interface | 3280 |
| remote external communication network | 3282 |
| local external communication network | 3284 |
| remote external device | 3286 |
| local external device | 3288 |
| output device | 3290 |
| display driver | 3292 |
| Display | 3294 |
| Humidifier | 4000 |
| humidifier inlet | 4002 |
| humidifier outlet | 4004 |
| humidifier base | 4006 |
| Reservoir | 4110 |
| conductive portion | 4120 |
| humidifier reservoir dock | 4130 |
| locking lever | 4135 |
| water level indicator | 4150 |
| heating element | 4240 |
| patient tracker/tracking device | 5000 |
| Patient | 5002 |
| body of the patient tracking device | 5004 |
| ear hook of the patient tracking device | 5006 |
| Speaker | 5008 |
| patient interface | 5010 |
| Conduit | 5012 |
| Mask | 5014 |
| Respiratory therapy system | 6000 |
| Processor arrangement of the Respiratory therapy system | 6002 |
| Memory | 6004 |
| Communication module | 6006 |
| Respiratory therapy delivery arrangement | 6008 |

1-37. (canceled)

38. A wearable device for use with a respiratory therapy system, the device comprising a body configured to locate with respect to an ear of a patient under respiratory therapy and including at least one sensor to obtain sensor data including physiological data of the patient when so located, the wearable device being able to be worn by the patient independently of the respiratory therapy system and the at least one sensor being configured to acquire the physiological data during an off therapy period, the device being configured to provide the sensor data to a processor to aid in respiratory therapy of the patient under the respiratory therapy system.

39. A wearable device according to claim 38, wherein the sensor data further comprises activity data of the patient and/or environmental data surrounding the patient.

40. A wearable device according to claim 38, wherein the at least one sensor is further configured to acquire physiological data during an on-therapy period.

41. A wearable device according to claim 38, wherein the device is configured to provide the sensor data to a processor of the respiratory therapy system to effect change to the respiratory therapy.

42. A wearable device according to claim 38, wherein the sensor data is provided to the processor to determine one or more characteristics of the patient related to the respiratory therapy.

43. A wearable device according to claim 38, wherein the processor is configured to determine a correlation between the sensor data of the patient and the respiratory therapy of the patient, and wherein the wearable device is configured to receive and communicate information relating to the correlation to the patient to aid in the respiratory therapy of the patient.

44. A wearable device according to claim 38, wherein the body of the device is configured to locate with respect to an external auditory canal of the patient.

45. A wearable device according to claim 38, wherein the at least one sensor is configured with respect to the body to be:
i. in contact with the patient's skin for collecting physiological data of the patient; and/or
ii. spaced from the patient's skin for collecting environmental data related to the patient.

46. A wearable device according to claim 38, further comprising an audio unit operable to selectively generate at least one sound, the wearable device further comprising an ear hook configured to locate with respect to the patient's external ear, wherein the ear hook includes the at least one sensor.

47. A treatment system comprising:
a respiratory system comprising:
a flow generator configured to generate a flow of air;
a patient interface constructed and arranged to form a seal with a region of the patient's face surrounding an entrance to the patient's airways, the patient interface being configured to deliver the pressurised or breathable air to the patient's airways for respiratory therapy; and
an air delivery tube coupled between the flow generator and the patient interface to deliver the flow of air from the flow generator to the patient interface as the pressurised or breathable air; and
a wearable device configured to locate with respect to an ear of the patient, the device being as defined in claim 38 and being configured to provide sensor data to a processor to aid in respiratory therapy of the patient under the respiratory system.

48. A treatment system according to claim 47, wherein the respiratory system further comprises a processor to interact with the wearable device, the respiratory system processor configured to control an operation of the respiratory system responsive to the sensor data of the device.

49. A treatment system according to claim 47, wherein the sensor data is provided to the respiratory system processor to determine one or more characteristics of the patient.

50. A treatment system according to claim 47, wherein the respiratory system processor is arranged in the flow generator, the respiratory system processor controlling an operation of the flow generator.

51. A treatment system according to claim 47, wherein the flow generator comprises a display interface and the respiratory system processor is configured to generate an alert to be displayed on the display interface based on sensor data of the at least one sensor of the device.

52. A treatment system according to claim 47, wherein the wearable device is configured to provide sensor data to a processor of at least one supplementary computing device comprising a display interface configured to generate an alert to be displayed on the display interface of the supplementary computing device based on the sensor data of the device.

53. A treatment system according to claim 47, wherein the wearable device is operative to generate an alert to the patient responsive to an instruction from the processor, and wherein the processor is configured to establish at least one correlation between the one or more characteristics of the patient and the respiratory therapy of the patient, the correlation being configured to aid in the respiratory therapy of the patient, the alert generated being based on the at least one correlation.

54. A method of determining an adjustment to a respiratory therapy for a patient, comprising:
acquiring sensor data during an off therapy period using one or more sensors in a wearable device adapted to be located in an ear of the patient;
storing the acquired sensor data in a memory device;
determining an analysis data comprising one or more physiological or activity parameters, based on the stored data;
determining the adjustment on the basis of the analysis data.

55. A method according to claim 54, comprising acquiring a first sensor data during a first period of time and acquiring a second sensor data during a second period of time, wherein determining the analysis data comprises comparing the first sensor data with the second data, or comparing values of one or more parameters calculated from the first sensor data against values of the parameters calculated from the second sensor data.

56. A method according to claim 55, wherein the first time period is during the off-therapy period, and a second time period is during an on-therapy period.

57. A method according to claim 54, wherein determining the analysis data comprises modulating the physiological or activity parameters based on one or more environmental data.
